(19) United States
(12) Patent Application Publication
(10) Pub. No.: US 2024/0293618 A1
(43) Pub. Date: Sep. 5, 2024

(54) DETERMINATION OF DOSES OF INSULIN AND RELATED SYSTEMS, METHODS, AND DEVICES

(71) Applicant: Bigfoot Biomedical, Inc., Milpitas, CA (US)

(72) Inventors: Adnan JAFAR, Montreal (CA); Ahmad HAIDAR, Montreal (CA); Michael TSOUKAS, Pointe-Claire (CA)

(21) Appl. No.: 18/438,058

(22) Filed: Feb. 9, 2024

Related U.S. Application Data
(60) Provisional application No. 63/484,387, filed on Feb. 10, 2023.

(51) Int. Cl. A61M 5/172

(52) U.S. Cl. CPC A61M 5/1723

(57) ABSTRACT

Determination of bolus doses and/or bolus doses of insulin and related systems, methods, and devices are disclosed. A method of determining an insulin dose for a meal bolus, correction bolus, and/or basal may include: tracking variations in carbohydrate ratios (CRs) utilizing a Q-learning algorithm, tracking variations in correction factors (CFs) utilizing a nearest-neighbors Q-learning algorithm, and determining a dose for a meal bolus responsive to the tracked CRs and the tracked CFs.

### CROSS-REFERENCE TO RELATED APPLICATIONS

This application claims the benefit of U.S. Provisional Application No. 63/484,387, filed Feb. 10, 2023, which is incorporated herein by reference in its entirety.

### TECHNICAL FIELD

This disclosure relates generally to determination of bolus doses of rapid acting insulin and/or for basal doses of long acting insulin, and more particularly to the use of reinforcement learning to determine doses for bolus doses of rapid acting insulin and/or basal doses of long acting insulin as part of an insulin therapy to treat diabetes.

### BACKGROUND

Diabetes mellitus is a chronic metabolic disorder caused by the inability of a person's pancreas to produce sufficient amounts of the hormone insulin such that the person's metabolism is unable to provide for the proper absorption of sugar and starch. The inability to absorb those carbohydrates sometimes leads to hyperglycemia, i.e., the presence of an excessive amount of glucose within the blood plasma. Hyperglycemia has been associated with a variety of serious symptoms and life-threatening long-term complications such as dehydration, ketoacidosis, diabetic coma, cardiovascular diseases, chronic renal failure, retinal damage and nerve damages with the risk of amputation of extremities.

Often, a permanent therapy is necessary to maintain a proper glucose level within normal limits. Maintaining a proper glucose level is conventionally achieved by regularly supplying insulin to a person with diabetes (PWD). Maintaining a proper glucose level may create a significant cognitive burden for a PWD (or a caregiver) and affect many aspects of the PWD's life. For example, the cognitive burden on a PWD may be attributed to, among other things, tracking meals and constant check-ins and minor course corrections of glucose levels. The adjustments of glucose levels by a PWD may include taking insulin, tracking insulin dosing and glucose, deciding how much insulin to take, how often to take it, where to inject the insulin, and how to time insulin doses in relation to meals and/or glucose fluctuations.

Treatment plans may be difficult to implement because of, among other things, differences between how different individuals react to treatments, and in fluctuations in how individuals themselves react to treatments.

### SUMMARY

The present disclosure provides one or more computer-readable storage media and a method of determining an insulin dose for a meal bolus as defined in the appended claims. Determination of bolus doses and/or bolus doses of insulin and related systems, methods, and devices are disclosed. A method of determining an insulin dose for a meal bolus, correction bolus, and/or basal may include: tracking variations in carbohydrate ratios (CRs) utilizing a Q-learning algorithm, tracking variations in correction factors (CFs) utilizing a nearest-neighbors Q-learning algorithm, and determining a dose for a meal bolus responsive to the tracked CRs and the tracked CFs.

### BRIEF DESCRIPTION OF THE DRAWINGS

While this disclosure concludes with claims particularly pointing out and distinctly claiming specific embodiments, various features and advantages of embodiments within the scope of this disclosure may be more readily ascertained from the following description when read in conjunction with the accompanying drawings, in which:

FIG. 1 is a flowchart illustrating a method of determining an insulin dose for a meal bolus, correction bolus, and/or basal dose according to some embodiments.

FIG. 2 is a flowchart illustrating a learning method for carbohydrate ratios (CRs), according to some embodiments.

FIG. 3 is a flowchart illustrating a learning method for correction factors (CFs), according to some embodiments.

FIG. 4 is a flowchart illustrating a method for estimating a bolus dose and/or basal dose, according to some embodiments.

FIG. 5 is a block diagram of an architecture of a system for an advanced bolus calculator for PWDs on multiple daily injections (MDI) therapy, according to some embodiments.

FIG. 6 is a block diagram of a medical treatment system, according to some embodiments.

FIG. 7 illustrates a percentage time spent in target range, below 4.0 mmol/L, and above 10.0 mmol/L with the nominal scenario (mean±SD).

FIG. 8 illustrates a percentage time spent in target range, below 4.0 mmol/L, and above 10.0 mmol/L with the variance scenario (mean±SD).

FIG. 9 is a block diagram of circuitry that, in some embodiments, may be used to implement various functions, operations, acts, processes, and/or methods disclosed herein.

### DETAILED DESCRIPTION

In the following detailed description, reference is made to the accompanying drawings, which form a part hereof, and in which are shown, by way of illustration, specific examples of embodiments in which the present disclosure may be practiced. These embodiments are described in sufficient detail to enable a person of ordinary skill in the art to practice the present disclosure. However, other embodiments enabled herein may be utilized, and structural, material, and process changes may be made without departing from the scope of the disclosure.

The illustrations presented herein are not meant to be actual views of any particular method, system, device, or structure, but are merely idealized representations that are employed to describe the embodiments of the present disclosure. In some instances similar structures or components in the various drawings may retain the same or similar numbering for the convenience of the reader; however, the similarity in numbering does not necessarily mean that the structures or components are identical in size, composition, configuration, or any other property.

The following description may include examples to help enable one of ordinary skill in the art to practice the disclosed embodiments. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

It will be readily understood that the components of the embodiments as generally described herein and illustrated in the drawings could be arranged and designed in a wide variety of different configurations. Thus, the following description of various embodiments is not intended to limit the scope of the present disclosure, but is merely representative of various embodiments. While the various aspects of the embodiments may be presented in the drawings, the drawings are not necessarily drawn to scale unless specifically indicated.

Furthermore, specific implementations shown and described are only examples and should not be construed as the only way to implement the present disclosure unless specified otherwise herein. Elements, circuits, and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

Those of ordinary skill in the art will understand that information and signals may be represented utilizing any of a variety of different technologies and techniques. Some drawings may illustrate signals as a single signal for clarity of presentation and description. It will be understood by a person of ordinary skill in the art that the signal may represent a bus of signals, wherein the bus may have a variety of bit widths and the present disclosure may be implemented on any number of data signals including a single data signal.

The various illustrative logical blocks, modules, and circuits described in connection with the embodiments disclosed herein may be implemented or performed with a general purpose processor, a special purpose processor, a digital signal processor (DSP), an Integrated Circuit (IC), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor (may also be referred to herein as a host processor or simply a host) may be a microprocessor, but in the alternative, the processor may be any conventional processor, controller, microcontroller, or state machine. A processor may also be implemented as a combination of computing devices, such as a combination of a DSP and a microprocessor, a plurality of microprocessors, one or more microprocessors in conjunction with a DSP core, or any other such configuration. A general-purpose computer including a processor is considered a special-purpose computer while the general-purpose computer is configured to execute computing instructions (e.g., software code) related to embodiments of the present disclosure.

The embodiments may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media (e.g., non-transitory computer-readable media, without limitation) and communication media including any medium that facilitates transfer of a computer program from one place to another.

Any reference to an element herein utilizing a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. In addition, unless stated otherwise, a set of elements may include one or more elements.

As used herein, the term “substantially” in reference to a given parameter, property, or condition means and includes to a degree that one of ordinary skill in the art would understand that the given parameter, property, or condition is met with a small degree of variance, such as, for example, within acceptable manufacturing tolerances. By way of example, depending on the particular parameter, property, or condition that is substantially met, the parameter, property, or condition may be at least 90% met, at least 95% met, or even at least 99% met.

Some persons with diabetes (PWDs) on multiple insulin injections therapy use carbohydrate ratios (CRs) and correction factors (CFs) to determine mealtime insulin and correction boluses. The PWDs' physiological characteristics represented by CRs and CFs vary over time due to physiological changes in individuals' response to insulin. Tracking variations in a PWD's CRs and CFs is thus relevant to calculate insulin boluses.

Various embodiments disclosed herein implement a novel learning method that uses Q-learning (e.g., a model free reinforcement learning method, without limitation) to track CRs (e.g., optimal CRs, without limitation) and uses nearest-neighbors Q-learning to track CFs (e.g., optimal CFs, without limitation). The learning method was compared with a run-to-run and Herrero et al.'s algorithms (proposed in P. Herrero et al., “Method for automatic adjustment of an insulin bolus calculator: In silico robustness evaluation under intra-day variability,” Computer Methods and Programs in Biomedicine, vol. 119, no. 1, pp. 1-8, 2018 and P. Herrero et al., “Advanced Insulin Bolus Advisor Based on Run-To-Run Control and Case-Based Reasoning,” IEEE J Biomed Health Inform, vol. 19, no. 3, pp. 1087-96, 2015) over an 8-week period utilizing a validated simulator with a realistic scenario created with suboptimal CRs and CFs values, carbohydrate counting errors, and random meals sizes at random ingestion times. From Week 1 to Week 8, the learning algorithm increased the percentage of time spent in target glucose range (3.9 to 10.0 mmol/L) from 51% to 64% compared to 61% and 58% with the run-to-run and the Herrero et al.'s algorithms, respectively. The learning method decreased the percentage of time spent below 4.0 mmol/L from 9% to 1.9% compared to 3.4% and 2.3% with the run-to-run and the Herrero et al.'s algorithms, respectively. Therefore, the learning methods disclosed may improve glucose control in PWDs.

Type 1 diabetes is characterized by the autoimmune destruction of pancreatic beta islets cells that secrete insulin, a hormone that regulates blood glucose levels via the suppression of hepatic glucose production and the promotion of glucose utilization by cells. Insulin replacement therapy that achieves tight glucose control reduces macro- and micro-vascular complications, but hypoglycemia remains the main hurdle to achieve tight glucose targets and most people with type 1 diabetes still have suboptimal glucose control. Multiple daily injections (MDI) and continuous subcutaneous insulin infusion via an insulin pump remain the standard of care for PWDs, with MDI being the most used therapy due to its lower cost and case of access.

As used herein, the term “MDI therapy” refers to the use of multiple (e.g., three to four, without limitation) insulin injections per day, including long- and rapid-acting forms of insulin. While long-acting insulin controls background glucose metabolism throughout the day and night, rapid-acting insulin is used for post-prandial glucose control and for corrections of hyperglycemia. Though some people on MDI therapy use fixed doses and scales for meal and correction boluses, a more precise method to calculate rapid-acting insulin doses is through carbohydrate ratios (CRs) and correction factors (CFs, also referred to as “insulin sensitivity factors”). CR determines the grams of carbohydrates covered by 1 unit of insulin bolus. CF determines the amount of glucose level's drop by 1 unit of insulin bolus. However, these CRs and CF are known to fluctuate within and between days due to physiological changes in individuals' response to insulin. This represents one of the obstacles to achieving optimal glucose control, leading to the use of periodic adjustments of CRs and CF by PWDs and their health care providers.

Several methods have been proposed to automatically optimize CRs for MDI therapy. Herrero et al. combined a run-to-run framework with a case-based reasoning approach that solves current situations by recalling similar past situations (proposed in P. Herrero et al., “Advanced Insulin Bolus Advisor Based on Run-To-Run Control and Case-Based Reasoning,” IEEE J Biomed Health Inform, vol. 19, no. 3, pp. 1087-96, 2015). This algorithm was tested clinically in a 6-week feasibility study in 10 adults (M. Reddy et al., “Clinical safety and feasibility of the advanced bolus calculator for type 1 diabetes based on case-based reasoning: a 6-week nonrandomized single-arm pilot study,” Diabetes Technol & Ther, vol. 18, no. 8, pp. 487-493, 2016). Patek et al. developed an algorithm that estimates glucose fluxes from glucose data, then retrospectively simulate the glucose trace under different insulin treatments to select the optimal one (S. D. Patek et al., “Retrospective optimization of daily insulin therapy parameters: control subject to a regenerative disturbance process,” in Proceedings of the International Federation of Automatic Control Conference, 2016, vol. 49, no. 7, pp. 773-778). This algorithm was tested in a short (48-hour) randomized clinical study in 24 adults (M. D. Breton et al., “Continuous Glucose Monitoring and Insulin Informed Advisory System with Automated Titration and Dosing of Insulin Reduces Glucose Variability in Type 1 Diabetes Mellitus,” Diabetes Technol Ther, vol. 20, no. 8, pp. 531-540, 2018). Tyler et al. developed an artificial intelligence-based decision support system and tested it on retrospective data collected from 25 adults over 28 days (N.S. Tyler et al., “An artificial intelligence decision support system for the management of type 1 diabetes,” Nature Metabolism, vol. 2, no. 7, pp. 612-619, 2020). El-Fathi et al. and Toffanin et al. developed run-to-run learning algorithms to adjust CRs (A. El Fathi et al., “A Model-Based Insulin Dose Optimization Algorithm for People with Type 1 Diabetes on Multiple Daily Injections Therapy,” IEEE Transactions on Biomedical Engineering, vol. 68, no. 4, pp. 1208-1219, 2020 and C. Toffanin et al., “Toward a run-to-run adaptive artificial pancreas: In silico results,” IEEE Transactions on Biomedical Engineering, vol. 65, no. 3, pp. 479-488, 2017). The El-Fathi et al.'s algorithm was tested in a pilot randomized parallel study of 11 days in 21 youths comparing the algorithm's adjustments with those of physicians (A. El Fathi et al., “A pilot non-inferiority randomized controlled trial to assess automatic adjustments of insulin doses in adolescents with type 1 diabetes on multiple daily injections therapy,” Pediatric Diabetes, vol. 21, no. 6, pp. 950-959, 2020). The feasibility of the Toffanin et al.'s algorithm was tested in a one-month study in 18 adults (M. Messori, et al., “Individually adaptive artificial pancreas in subjects with type 1 diabetes: a one-month proof-of-concept trial in free-living conditions,” Diabetes Technol. Ther, vol. 19, no. 10, pp. 560-571, 2017). Finally, Herrero et al. proposed a novel bolus calculator for CRs adjustments based on a run-to-run method (P. Herrero et al., “Method for automatic adjustment of an insulin bolus calculator: In silico robustness evaluation under intra-day variability,” Computer Methods and Programs in Biomedicine, vol. 119, no. 1, pp. 1-8, 2018). The bolus calculator proposed by Herrero et al. was tested utilizing computer simulations.

CRs adjustments according to these and other approaches may be suboptimal as they do not consider before and after meals' correction boluses. Also, these and other approaches adjust CRs only, and CF is calculated from the total daily insulin dose utilizing a static 100-rule equation, which was proposed in J. Walsh, R. Roberts, and T. Bailey, “Guidelines for optimal bolus calculator settings in adults,” Journal of Diabetes Science and Technology, vol. 5, no. 1, pp. 129-135, 2011. Although the 100-rule is widely used for CF estimation, data in children and adolescents show that the actual doses needed are on average stronger than those calculated by the 100-rule. Moreover, CF may be affected by several factors including puberty, age, and body mass index. Some studies also reported that CF may be different between boys and girls during puberty. In addition, diurnal variations in insulin sensitivity occur throughout the day due to the varying magnitude of different hormonal secretions. Therefore, tracking variations in CFs is also useful to calculate optimal insulin boluses.

In recent years, reinforcement learning has gained increased popularity to solve various problems such as medication dosing, autonomous driving, and the board-game “Go,” by way of non-limiting example.

Various embodiments disclosed herein include a novel learning method to simultaneously adjust CRs and CFs in PWDs on MDI therapy. A Q-learning approach may be used to adjust CRs with novel state and reward functions including the effect of before and after meals' correction boluses and the rate of change of late post-prandial glucose levels. To adjust CFs, a nearest-neighbors method with Q-learning may be used to have a finite sample convergence since pure correction boluses data are scarce. To account for intra-day variability, one CF for daytime and one CF for nighttime may be used. Learning methods according to various embodiments disclosed herein may be compared with the run-to-run and the Herrero et al.'s algorithms over an 8-week period utilizing a validated simulator with realistic scenarios.

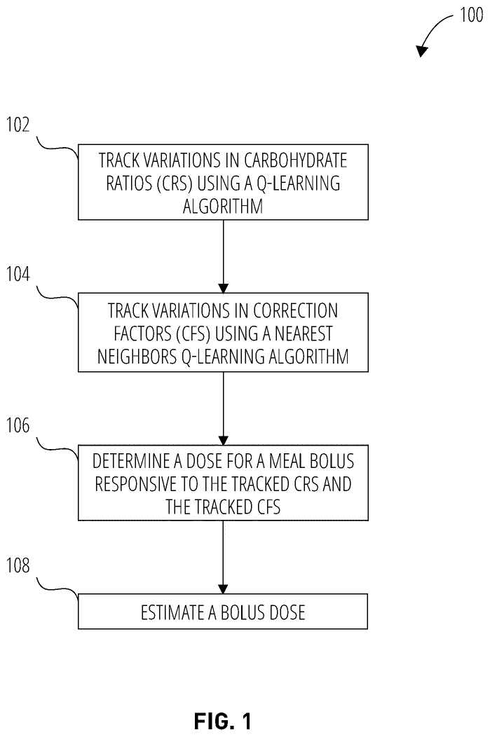

FIG. 1 is a flowchart illustrating a method 100 of determining an insulin dose for a meal bolus, a dose for a correction bolus, and/or a basal dose, according to some embodiments. At operation 102, method 100 includes tracking variations in CRs utilizing a Q-learning algorithm. At operation 104, method 100 includes tracking variations in CFs utilizing a nearest-neighbors Q-learning algorithm. At operation 106, method 100 includes determining a dose for a meal bolus responsive to the tracked CRs and the tracked CFs. At operation 108, method 100 includes estimating a dose.

### A. Bolus Calculation

A formula for calculating a bolus is as follows:

$$B = \frac{CHO}{CR} + \frac{{G}_{m} - {G}_{T}}{CF} - IOB \qquad ( 1 )$$

where Gₘ is the blood glucose level (mmol/L), G_{T} is the target glucose level (mmol/L), CHO is the amount of carbohydrates in the meal (g), and IOB is the insulin on board that is still working from previous insulin doses. By way of non-limiting example, formula (1) may be used at operation 106 of method 100 of FIG. 1 to determine a dose for a meal bolus.

### B. Problem Formulation

Reinforcement learning: A reinforcement learning framework may be assumed to be a discrete Markov decision process (S, A, P, r), with state space S, action space A, transition dynamics P(sₖ₊₁|sₖ, aₖ), and reward r. The agent receives a reward rₖ(sₖ, aₖ, sₖ₊₁)ϵ [symbol] after taking an action aₖ in a state sₖ and reaching at a state sₖ₊₁. The agent's goal is to maximize the long-term return:

$${R}_{k} = {\sum }_{i = k}^{∞} {γ}^{i - k} {r}_{i} ( {s}_{i} , {a}_{i} , {s}_{i + 1} ) \qquad ( 2 )$$

where γϵ[0, 1) is a discount factor that weights the preference of immediate (small γ) over future (large γ) rewards.

The agent selects its actions based on a policy π:S→A. The value function Jₖ^{π}:S*A→ [symbol] under a policy π can be described as the expected return in the future time T when the policy π is followed at the state sₖ:

$${J}_{k}^{π} ( {s}_{k} ) = {𝔼}_{π} [ {{\sum }^{k + T}}_{t = k} {γ}^{i - k} {r}_{i} \vert s = {s}_{k} ] \qquad ( 3 )$$

Utilizing a backward recursive equation, the value of Iₖ^{π}(sₖ) may be re-written as:

$${J}_{k}^{π} ( {s}_{k} ) = {𝔼}_{π} [ {r}_{k} + γ {{\sum }^{k + T}}_{i = k ← 1} {γ}^{i - ( k + 1 )} {r}_{i} \vert s = {s}_{k} ] = {\sum }_{{a}_{k} ϵ A ( {s}_{k} )} π ( {s}_{k} , {a}_{k} ) {\sum }_{{z}_{k} \text{?} ϵ S} p \text{?} [ {r}_{k} + {YJ}_{k + 1}^{π} ( {s}_{k \to 1} ) ] \qquad ( 4 )$$

$$\text{?} \text{indicates text missing or illegible when filed}$$

where pₛ_{ₖ}ₛ_{ₖ₊₁}^{a}^{ₖ} is the state transition probability from the state sₖ to the state sₖ₊₁ when action aₖ is taken.

Utilizing the Bellman optimality equation, one can find the maximum value function over the policy π as:

$${J}_{k}^{*} ( {s}_{k} ) = {max}_{π} {\sum }_{{a}_{k} ϵ A ( {s}_{k} )} π ( {s}_{k} , {a}_{k} ) {\sum }_{{s}_{k + 1} ϵ S} {p}_{{s}_{k} {s}_{k + 1}}^{{a}_{k}} [ {r}_{k} + {YJ}_{k + 1}^{*} ( {s}_{k + 1} ) ] \qquad ( 5 )$$

An optimal policy at time step k is defined as

$${π}^{*} ( {s}_{k} ) = arg {max}_{π} {J}_{k}^{*} ( {s}_{k} ) .$$

An optimal action-value function Qₖ*(sₖ, aₖ) is defined as the expected return obtained when π*(s) is followed thereafter:

$${Q}_{k}^{*} ( {s}_{k} , {a}_{k} ) = {\sum }_{{s}_{k + 1} ϵ S} {p}^{{a}_{k}} \text{?} [ {r}_{k} + {YJ}_{k + 1}^{*} ( {s}_{k + 1} ) ] \qquad ( 6 )$$

$$\text{?} \text{indicates text missing or illegible when filed}$$

In some embodiments, the model-free temporal-difference method may be used to estimate Qₖ*(sₖ, aₖ). In such embodiments the action-value function (5) may be re-written as:

$${Q}_{k + 1} ( {s}_{k} , {a}_{k} ) = {Q}_{k} ( {s}_{k} , {a}_{k} ) + α [ {r}_{k} + γ {max}_{{a}_{u}} {Q}_{k} ( {s}_{k + 1} , {a}_{u} ) - {Q}_{k} ( {s}_{k} , {a}_{k} ) ] \qquad ( 7 )$$

where αϵ[0, 1) is the learning rate and u is an index of action in the action space A.

Nearest-neighbors Q-learning: Let S be a compact state space and ρ be a metric in S. For every scalar h>0, a finite set of states

$${S}_{h} {=}^{△} {\{ {v}_{i} , {v}_{i} ∈ S \}}_{i = 1}^{n}$$

may be found that discretizes S utilizing h-covering balls centered at vᵢ:

$$∀ s ϵ S , ∃ {v}_{i} such that \qquad ( 8 )$$

$$ρ ( s , {v}_{i} ) < h$$

Supposing Q-values have been estimated for the set of states vᵢ denoted as Q={Q(vᵢ, a), vᵢ∈Sₕ, a∈A}, the Q-value for any state-action pair (s, a) may be estimated in S utilizing the weighted average of the Q-values of nearest-neighbors vᵢ as follows:

$$\hat{Q} ( s , a ) = {{\sum }^{n}}_{i = 1} W ( s , {v}_{i} ) Q ( {v}_{i} , a ) , ∀ s ∈ S , a ϵ A \qquad ( 9 )$$

where W is a weighting function satisfying

$${{\sum }^{n}}_{i = 1} W ( s , {v}_{i} ) = 1 , W ( s , {v}_{i} ) = 0 if ρ ( s , {v}_{i} ) \geq h \qquad ( 10 )$$

The Q-value of the state-action pair (vᵢ, a) may be estimated as follows:

$${Q}_{k + 1} ( {v}_{i} , a ) = ( 1 - {α}_{k} ) {Q}_{k} ( {v}_{i} , a ) + {α}_{k} {JQ}_{k} ( {v}_{i} , a ) \qquad ( 11 )$$

where αₖ∈[0, 1) is the learning rate and JQ is the joint nearest-neighbors Q-value operator for each state-action pair (vᵢ, a) as follows:

$${JQ}_{k} ( {v}_{i} , a ) = {r}_{k} ( {s}_{k} , a , {s}_{k + 1} ) + γ 𝔼 [ {max}_{b ∈ A} \hat{Q} ( {s}_{k + 1} , b ) \vert {v}_{i} , a ] \qquad ( 12 )$$

Unlike standard Q-learning, the Q-values of each state-action pair (vᵢ, a) is estimated utilizing all states that lie in its neighborhood.

To learn the optimal policy, the agent should visit the all state-actions pairs and improve its current policy by selecting actions that were tried in the past and contributed most to the accumulated rewards. One way to achieve this is to use the ε-greedy policy. The ϵ-greedy policy proposed, for example, in R.S. Sutton and A.G. Barto, “Reinforcement learning: An introduction,” 2011, in which the agent explores (tries new actions) with a probability ε and exploits (uses experience) with a probability 1−ε. The agent uses exploitation to take advantage of prior knowledge and exploration to identify new options. The agent chooses the optimal action to generate the maximum reward possible for a given state. During the learning period, the ε starts at 0.9 and gradually reduces to a small value 0.1.

### C. Learning Method for CRs

In this sub-section, the states, actions, and rewards are defined for the CRs learning algorithm.

### 1) State Space

The state sₖ^{m} for each meal type (breakfast, lunch, and dinner) includes (i) the glucose rate of change in the period between t₁ and t₂ after meals, ROCₖ^{p}, and (ii) the post-prandial glucose error defined as:

$${E}_{k} = {G}_{min} ( k ) - {G}_{T} \qquad ( 13 )$$

where G_{T} is the target glucose level and Gₘᵢₙ(k) is the minimum glucose level in the period between t₃ and t₄ after the meals. t₁, t₂, t₃, and t₄ are tuning parameters. In case of before- or after-meal correction bolus, the predicted glucose profile was used, calculated after each correction bolus utilizing a linear prediction model with parameters of current glucose level, and weighted average of the rate of change of two consecutive glucose values over the last 60 minutes window.

The value Pₖ^{hypo} is the percentage of time spent below 4.0 mmol/L in the period between 1 and 6 hours after the meals, then the goal state s_{g}^{m} may be defined as a rate of change (ROC) range |ROCₖ^{p}|≤ROC_{T}, an error range |Eₖ|≤E_{T} and a percent of time Pₖ^{hypo}=0%, where ROC_{T} and E_{T} are tuning parameters. This choice of the state representation and the goal state allows the method to aim for tight and stable late post-prandial glucose levels.

### 2) Action Space

Let A(s^{m}) be the set of all possible actions at state s^{m}. For all states, the action space A may be defined as follows:

$$A ( {s}^{m} ) = \{ {a}^{m} \vert 1 , - 1 , 0 \} \qquad ( 14 )$$

where 1, −1, 0 represent increasing, decreasing, and not changing CRs relative to previous day's values, respectively.

### 3) Reward Function

After taking an action aₖ^{m}, the agent receives a reward rₖ^{m} as follows:

$${r}_{k}^{m} = \{ \begin{matrix}10 {e}^{\frac{\vert Δ {E}_{k + 1} \vert}{10} + {R}_{m}} & if ( \vert {E}_{k + 1} \vert \leq \vert {E}_{k} \vert ) \& ( {ROC}_{k + 1}^{p} \leq {ROC}_{k}^{p} ) \\ 50 & if ( {s}_{k + 1}^{m} = {s}_{g}^{m} ) \\ 1 & if ( {s}_{k + 1}^{m} = {s}_{k}^{m} ) \\ - 10 {ne}^{\frac{\vert Δ {E}_{k + 1} \vert}{10}} & otherwise\end{matrix} \qquad ( 15 )$$

where n is a scaling multiplier, which equals 1 if glucose levels do not fall below 4.0 mmol/L in the post-prandial period, and which equals 2 otherwise. Rₘ is defined as:

$${R}_{m} = \frac{1}{M ( {s}_{k}^{m} , {a}_{k}^{m} )} \vert γ {max}_{{a}_{u}} {Q}_{0} ( {s}_{k + 1}^{m} , {a}_{u} ) - {Q}_{0} ( {s}_{k}^{m} , {a}_{k}^{m} ) \vert \qquad ( 16 )$$

and Mₖ^{m}, aₖ^{m}) is a counter that records how many times the state-action pair (sₖ^{m}, aₖ^{m}) has been visited.

The reward function (15) encourages the learning method to take the actions that do not increase the post-prandial glucose error Eₖ₊₁ and the glucose rate of change ROCₖ₊₁^{p}. If the actions led to the goal state s_{g}^{m} with no hypoglycemia, then the agent receives a large positive reward. If the action did not lead to a change in the state with no hypoglycemia, then the agent receives a constant reward of 1. In all other cases, the agent receives a negative reward. The boosting term Rₘ is included to give the learning agent additional reward during the initial learning phase by an offline-averaged reward from the state-action pair (sₖ^{m}, aₖ^{m}) to the next state sₖ₊₁^{m} for each successful action to promote early and safe convergence.

The details of the learning method for CR estimation are given in Algorithm 1.

Algorithm 1: Learning method for CRs

1. Initialize γ = 0.9, α = 0.55, ε = 0.9.
2. Initialize Q(s^{m}, ^{m}), ∀s^{m} ∈ S^{m}, α^{m} ∈ S utilizing clinical guidelines.
3. Evaluate current state sₖ^{m},
4. Repeat
5. Choose action aₖ^{m} from sₖ^{m} utilizing _{ε}-greedy policy.
6. From an action aₖ^{m}, obtain CRₖ₊₁ utilizing the following:
$C {R}_{k + 1} = \{ \begin{matrix}C {R}_{k} + {k}_{1} C {R}_{k} & if {P}_{k}^{h y p o} > 5 \% \\ C {R}_{k} + {k}_{2} C {R}_{k} & if 0 \% < {P}_{k}^{h y p o} \leq 5 \% \\ C {R}_{k} + {a}_{k}^{m} {k}_{h} C {R}_{k} , ∀ {a}_{k}^{m} ϵ A & if {P}_{k}^{h y p o} = 0 \%\end{matrix}$
7. Apply CRₖ₊₁, observer the reward rₖ^{m} and the next state sₖ₊₁^{m}.
8. Update the action-value function (7).
9. sₖ^{m} ← sₖ₊₁^{m}.

k₁=0.2, k₂=0.15, and kₕ=(0.05Eₖ−0.05) were chosen empirically utilizing clinical guidelines and targeting a convergence in 7 iterations.

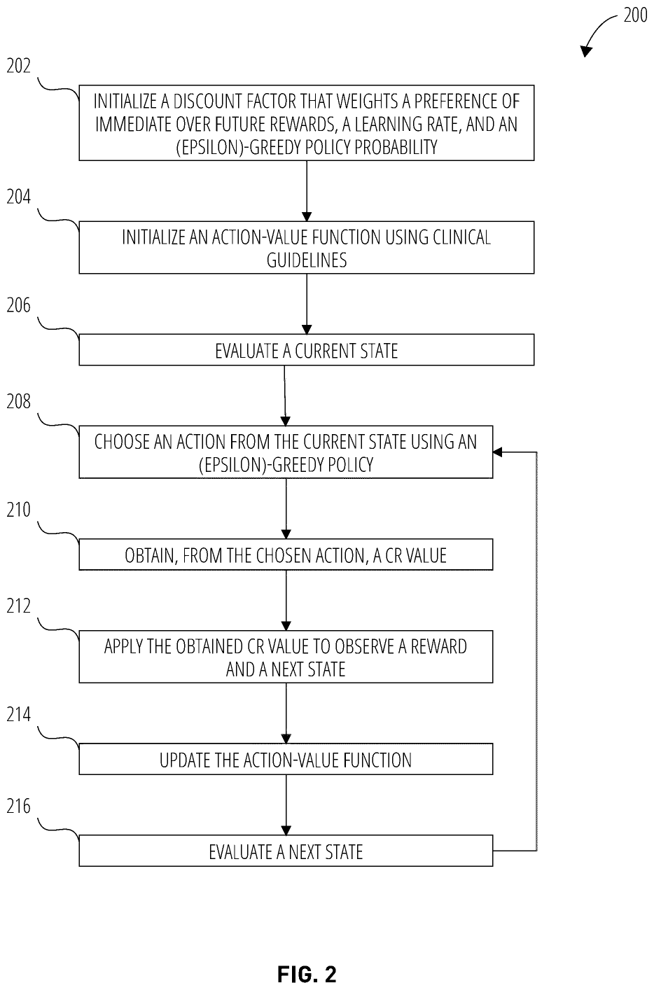

FIG. 2 is a flowchart illustrating a learning method 200 for CRs, according to some embodiments. By way of non-limiting example, the learning method 200 may be used in operation 102 of FIG. 1 to track the variations in CRs. Algorithm 1, discussed above, is an example of the learning method 200.

At operation 202, learning method 200 includes initializing a discount factor that weights a preference of immediate over future rewards, a learning rate, and an ε-greedy policy probability. At operation 204, learning method 200 includes initializing an action-value function utilizing clinical guidelines. At operation 206, learning method 200 includes evaluating a current state.

Learning method 200 repeats operation 208, operation 210, operation 212, operation 214, and operation 216. At operation 208, the learning method 200 includes choosing an action from the current state utilizing an ε-greedy policy. At operation 210, learning method 200 includes obtaining, from the chosen action, a CR value. At operation 212, learning method 200 includes applying the obtained CR value to observe a reward and a next state. At operation 214, learning method 200 includes updating the action-value function. At operation 216, learning method 200 includes evaluating a next state. Learning method 200 may return to operation 208 for the next state as the current state.

### D. Learning Method for CFs

In this sub-section, a learning method for CFs estimation is disclosed. One CF for daytime (7 AM to 12 AM) and one CF for nighttime (12 AM to 7 AM) are disclosed. The CFs for daytime and nighttime are estimated utilizing a nearest-neighbors Q-learning algorithm. The nearest-neighbors with the Q-learning approach may be chosen to have finite sample convergence since the pure (i.e., not accompanied with a meal bolus) correction boluses data are scarce.

### 1) State Space

The state sₖ^{c} for CFs learning algorithm in an interval I includes (i) the glucose rate of change at the correction bolus time and in the period between t₁ and t₂ after the correction bolus, ROC_{k,c}^{c} and ROC_{k,c}^{p}, respectively (ii) the glycemic risk index GRI_{k,c}, calculated as a linear combination of hypoglycemia and hyperglycemia components, and (iii) the post-correction glucose error defined as:

$${E}_{k}^{′} = {G}_{min , c} ( k ) - {G}_{T} \qquad ( 17 )$$

where G_{T} is the target glucose level and G_{min,c}(k) is the minimum glucose level in the period between t₃ and t₄ after the correction bolus. t₁, t₂, t₃, and t₄ are tuning parameters.

The value P_{k,c}^{hypo} may be the percentage of time spent below 4.0 mmol/L in the period between 1 and 6 hours after the correction bolus, the goal state s_{g}^{c} was defined as |ROC_{k,c}^{p}|≤ROC_{T}, |Eₖ′|≤E_{T} and P_{k,c}^{hypo}=0%, where ROC_{T} and E_{T} are tuning parameters. The choice of the state representation and the goal state is the same as discussed above for the CRs learning algorithm plus the added glycemic risk index parameter to track the overall quality of glycemia in the interval I.

### 2) Action Space

The set A(s^{c}) is the set of all possible actions at state, s^{c}. For all states, the action space A is defined as follows:

$$A ( {s}^{c} ) = \{ {a}^{c} \vert 1 , - 1 , 0 \} \qquad ( 18 )$$

where 1, −1,0 represent increasing, decreasing, and not changing CFs relative to previous day's values, respectively.

### 3) Reward Function

Responsive to taking an action aₖ^{c}, the agent receives a reward rₖ^{c} as follows:

$${r}_{k}^{c} = \{ \begin{matrix}10 {r}_{c} {e}^{\frac{\vert Δ {E}_{k + 1}^{′} \vert}{10} + {R}_{c}} & if ( \vert {E}_{k + 1}^{′} \vert \leq \vert {E}_{k}^{′} \vert ) \& ( {ROC}_{k + 1 , c}^{p} \leq {ROC}_{k , c}^{p} ) \\ 50 & if ( {s}_{k + 1}^{c} = {s}_{g}^{c} ) \\ 1 & if ( {s}_{k + 1}^{c} = {s}_{k}^{c} ) \\ - 10 {nr}_{c} {e}^{\frac{\vert Δ {E}_{k + 1}^{′} \vert}{10}} & otherwise\end{matrix} \qquad ( 19 )$$

where n is a scaling multiplier equal to 1 if glucose levels do not fall below 4.0 mmol/L in the post-correction period and is equal to 2 otherwise. R_{c} and r_{c} are defined as:

$${R}_{c} = \frac{1}{N ( {s}_{k}^{c} , {a}_{k}^{c} )} \vert γ {max}_{{a}_{u}} {Q}_{0} ( {s}_{k + 1}^{c} , {a}_{u} ) - {Q}_{0} ( {s}_{k}^{c} , {a}_{k}^{c} ) \vert \qquad ( 20 )$$

$${r}_{c} = 1 - min ( \frac{{GRI}_{k , c}}{100} , 0.5 ) \qquad ( 21 )$$

where N(sₖ^{c}, aₖ^{c}) is a counter that records how many times the state-action pair (sₖ^{c}, aₖ^{c}) has been visited.

The reward function (19) encourages the learning method to take the actions that does not increase the post-correction glucose error Eₖ₊₁′ and the glucose rate of change ROC_{k+1,c}^{p} while taking into account the GRI_{k,c} in the interval I. If the actions led to the goal state s_{g}^{c} with no hypoglycemia, then the agent receives a large positive reward. If the action did not lead to a change in the state with no hypoglycemia, then the agent receives a constant reward of 1. In all other cases, the agent receives a negative reward. The boosting term R_{c} was added to give the learning agent an extra reward during the initial learning phase by an offline averaged reward from the state-action pair (sₖ^{c}, aₖ^{c}) to the next state sₖ₊₁^{c} for each successful action to promote early and safe convergence.

The details of the learning method for CFs estimation are given in Algorithm 2.

Algorithm 2: Learning method for CFs

```
  1. Initialize γ = 0.9, ε = 0.9.
  2. Construct finite set of state space S [symbol] ; k = t = 0, [symbol]  = 0.
  3. Initialize Q(vᵢ, [symbol] ), ∀vᵢ ∈ Sₕ, [symbol]  ∈ A utilizing clinical guidelines.
  4. For each (vᵢ, [symbol] ), set the counter value Nₒ ( vᵢ, [symbol] ) = 0.
  5. Evaluate current state sₖ [symbol]
  6. Repeat
  7. Choose action [symbol]  from [symbol]  utilizing _{ε}-greedy policy.
  8. From an action [symbol] , obtain CFₖ₊₁, utilizing the following:
    $C {F}_{k + 1} = \{ \begin{matrix}C {F}_{k} + {k}_{c 1} C {F}_{k} & if {P}_{k , c}^{h y p o} > 5 \% \\ {CF}_{k} + {k}_{c 2} C {F}_{k} & if 0 \% < {P}_{k , c}^{h y p o} \leq 5 \% \\ C {F}_{k} + {a}_{k}^{c} {k}_{c 3} C {F}_{k} & if {P}_{k , c}^{h y p o} = 0 \%\end{matrix}$
  9. Apply CFₖ₊₁, observe the reward [symbol]  and the next state sₖ₊₁ [symbol] .
10. For each state vᵢ, nearest to sₖ [symbol]  do
    If Nₖ(vᵢ, sₖ [symbol] ) > 0
$J {Q}_{k} ( {v}_{i} , {a}_{k}^{c} ) = ( 1 - \frac{1}{1 + {N}_{k} ( {v}_{i} , {a}_{k}^{c} )} ) {JQ}_{k} ( {v}_{i} , {a}_{k}^{c} ) + \frac{1}{1 + {N}_{k} ( {v}_{i} , {a}_{k}^{c} )} ( {r}_{k}^{c} + γ {max}_{b ∈ a} {\hat{Q}}_{k} ( {s}_{k + 1}^{c} \text{?} .$
Else
${JQ}_{k} ( {v}_{i} , {a}_{k}^{c} ) = \text{?} + γ {max}_{b ∈ A} {\hat{Q}}_{k} ( \text{?} , b ) .$
End
Nₖ(vᵢ, [symbol] ) = Nₖ(vᵢ, [symbol] ) + 1.
End
11. For each state vᵢ, nearest to s [symbol]
Qₖ₊₁(υᵢ, αₖ^{c}) = (1 − αₖ(υᵢ, αₖ^{c}))Qₖ(υᵢ, αₖ^{c}) + αₖ(υᵢ, αₖ^{c})JQₖ(υᵢ, αₖ^{c}).
${α}_{k} ( {v}_{i} , \text{?} ) = \frac{β}{\text{?} + {N}_{k} ( {v}_{i} \cdot \text{?} )} .$ [symbol]
indicates data missing or illegible when filed
```

k_{c1}=0.2, k_{c2}=0.15, and k_{c3}=(0.05E_{k,c}−0.05) were chosen empirically utilizing clinical guidelines and targeting an algorithm's convergence in 7 iterations *Nₖ(vᵢ, aₖ^{c}) is the counter that records how many times the state-action pair (vᵢ, aₖ^{c}) has been visited.

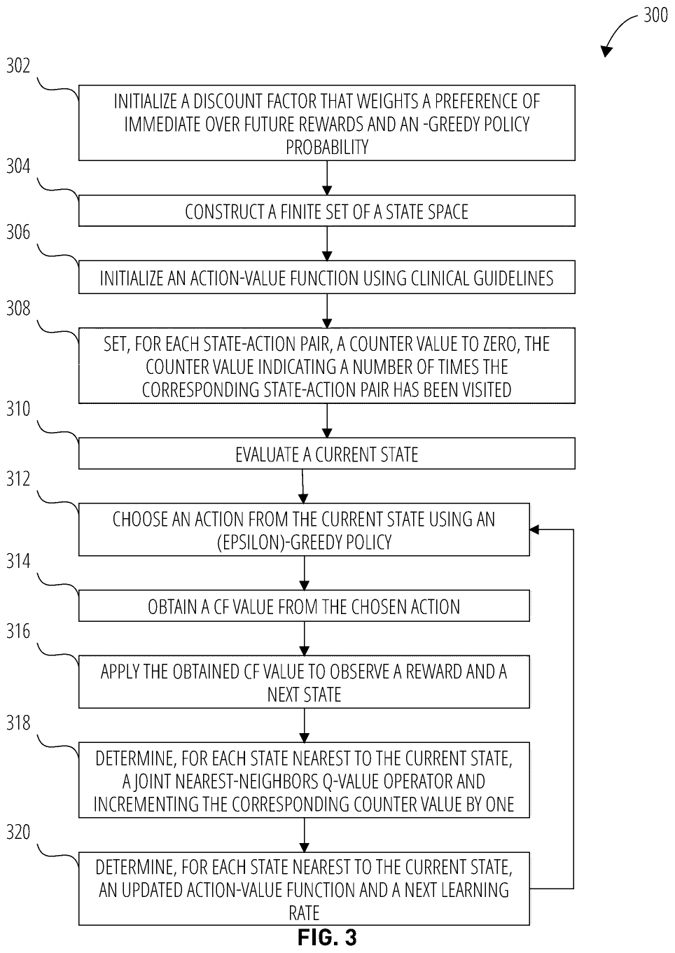

FIG. 3 is a flowchart illustrating a learning method 300 for CFs, according to some embodiments. By way of non-limiting example, the learning method 300 may be used in operation 104 of FIG. 1 to track the variations in CFs. Algorithm 2, discussed above, is an example of the learning method 300.

At operation 302, learning method 300 includes initializing a discount factor that weights a preference of immediate over future rewards and an ε-greedy policy probability. At operation 304, learning method 300 includes constructing a finite set of a state space. At operation 306, learning method 300 includes initializing an action-value function utilizing clinical guidelines. At operation 308, learning method 300 includes setting, for each state-action pair, a counter value to zero, the counter value indicating a number of times the corresponding state-action pair has been visited. At operation 310, learning method 300 includes evaluating a current state.

Learning method 300 repeats operation 312, operation 314, operation 316, operation 318, and operation 320. At operation 312, learning method 300 includes choosing an action from the current state utilizing an ε-greedy policy. At operation 314, learning method 300 includes obtaining a CF value from the chosen action. At operation 316, learning method 300 includes applying the obtained CF value to observe a reward and a next state. At operation 318, learning method 300 includes determining, for each state nearest to the current state, a joint nearest-neighbors Q-value operator and incrementing the corresponding counter value by one. At operation 320, learning method 300 includes determining, for each state nearest to the current state, an updated action-value function and a next learning rate. Learning method 300 may return to operation 312 for the next state as the current state.

### E. Learning Algorithm for Basal

In this sub-section, the states, actions, and rewards are defined for the basal learning algorithm.

### 1) State Space

The state sₖ^{b} for the basal learning method includes (i) the mean glucose error Uₖ, calculated as the mean glucose Gₘₑₐₙ in the period between 2:00 a.m. and 7:00 a.m. minus the target glucose level G_{T}, (ii) the percentages of time spent below 4.0 and above 10.0 mmol/L, Tₖ^{hypo} and Tₖ^{hyper}, respectively, in the period between 2:00 a.m. and 7:00 a.m., (iii) the rate of glucose change in the period between 4:00 a.m. and 7:00 a.m., and (iv) the hypoglycemia treatment in the period between 10:00 p.m. and 2:00 a.m. The goal state s_{g}^{b} may be defined to be

$$\vert {U}_{k} \vert \leq \frac{1 m mol}{L} ,$$

Tₖ^{hypo}=0, Tₖ^{hyper}=0.

The choice of the state representation and the goal state allows the algorithm to aim for tight and stable fasting glucose levels.

### 2) Action Space

The set A(s^{b}) may be the set of all possible actions at state, s^{b}. For all states, the action space A may be defined as follows:

$$A ( {s}^{b} ) = \{ {a}^{b} \vert 1 , - 1 , 0 \} \qquad ( 22 )$$

where 1, −1, 0 represent increasing, decreasing, and not changing basal relative to previous day's values, respectively.

### 3) Reward Function

Responsive to taking an action aₖ^{b}, the agent receives a reward rₖ^{b}.

$$( 23 )$$

$${r}_{k}^{b} = \{ \begin{matrix}10 {e}^{\frac{\vert Δ {U}_{k + 1} \vert}{10} + {R}_{b}} & if ( {T}_{k + 1}^{hyper} \leq {T}_{k}^{hyper} ) \& ( {U}_{k} \leq {U}_{k - 1} ) \& ( {T}_{k + 1}^{hypo} \leq {T}_{k}^{hypo} ) \\ 50 & if ( {s}_{k + 1}^{b} = {s}_{g}^{b} ) \\ 1 & if ( {s}_{k + 1}^{b} = {s}_{k}^{b} ) \\ - {e}^{\frac{\vert Δ {U}_{k + 1} \vert}{10}} & otherwise\end{matrix}$$

where n is a scaling multiplier equal to 1 if glucose levels do not fall below 4.0 mmol/L in the period between 2:00 a.m. and 7:00 a.m. and equal to 2 otherwise. R_{b} is defined as:

$${R}_{b} = \frac{1}{O ( {s}_{k}^{m} , {a}_{k}^{m} )} \vert γ {max}_{{a}_{u}} {Q}_{0} ( {s}_{k + 1}^{b} , {a}_{u} ) - {Q}_{0} ( {s}_{k}^{b} , {a}_{k}^{b} ) \vert \qquad ( 24 )$$

and O(sₖ^{b}, aₖ^{b}) is the counter that records how many times the state-action pair (sₖ^{b}, aₖ^{b}) has been visited.

The reward function (23) encourages the agent to take actions that do not increase mean glucose error ΔUₖ, Tₖ^{hypo}, and Tₖ^{hyper}. If the selected action led to the goal state s_{g}^{b}, then the agent receives a large positive reward. If the selected action did not lead to change in the state, then the agent receives a constant reward of 1. In all other cases, the agent receives a negative reward.

The details of the learning method for basal estimation are given in Algorithm 3.

Algorithm 3: Learning method for basal

1. Initialize γ = 0.9, α = 0.55, ε = 0.9.
2. Initialize Q(s^{b}, a^{b}), ∀s^{b} ∈ S^{b}, ∀a^{b} ∈ A utilizing clinical guidelines.
3. Evaluate current state sₖ^{b}.
4. Repeat
5. Choose action aₖ^{b} from sₖ^{b} utilizing _{ε}-greedy policy.
6. From an action aₖ, obtain Bₖ₊₁ utilizing the following:
${B}_{k + 1} = \{ \begin{matrix}{B}_{k} - {k}_{1} {B}_{k} & if {T}_{k}^{hypo} > 5 \% \\ {B}_{k} - {k}_{2} {B}_{k} & if 0 \% < {T}_{k}^{hypo} \leq 5 \% \\ {B}_{k} + {a}_{k}^{b} {k}_{1} {B}_{k} , ∀ {a}_{k}^{b} ϵ A & if {T}_{k}^{hypo} = 0 \%\end{matrix}$
7. Apply Bₖ₊₁, observe the reward rₖ^{b} and the next state sₖ₊₁^{b},
8. Update the action-value function (7).
9. sₖ^{b} ← sₖ₊₁^{b}.

k₁=0.2, k₂=0.15, and

$${k}_{1} = 0.1 \frac{{G}_{mean} - {G}_{T}}{10 - 4}$$

were chosen empirically utilizing clinical guidelines and targeting an algorithm's convergence in seven iterations.

To safely use the learning method and to ensure its robustness in abnormal days, updates in CRs, CFs, and basal values are restricted to ±20% of previous day's values.

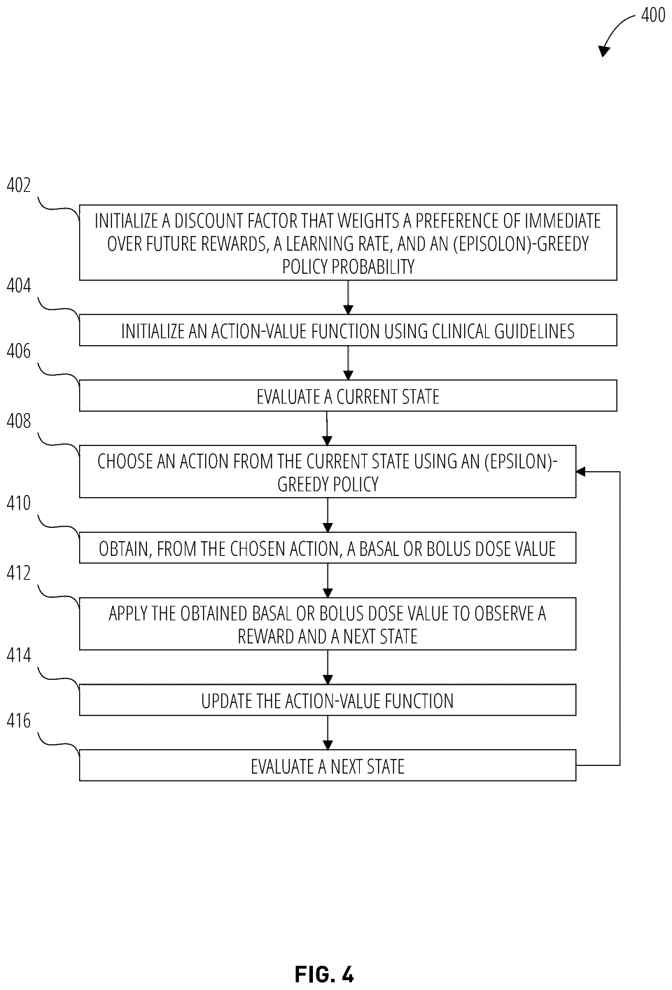

FIG. 4 is a flowchart illustrating a method 400 for estimating a basal dose, according to some embodiments. By way of non-limiting example, the method 400 may be used in operation 108 of FIG. 1 to estimate a dose for a bolus or basal dose. Algorithm 3, discussed above, is an example of the method 400.

At operation 402, method 400 includes initializing a discount factor that weights a preference of immediate over future rewards, a learning rate, and an ε-greedy policy probability. At operation 404, method 400 includes initializing an action-value function utilizing clinical guidelines. At operation 406, method 400 includes evaluating a current state.

Method 400 repeats operation 408, operation 410, operation 412, operation 414, and operation 416. At operation 408, method 400 includes choosing an action from the current state utilizing an ε-greedy policy. At operation 410, method 400 includes obtaining, from the chosen action, a basal or bolus dose value. At operation 412, method 400 includes applying the obtained basal or bolus dose value to observe a reward and a next state. At operation 414, method 400 includes updating the action-value function. At operation 416, method 400 includes evaluating a next state.

### F. System Architecture of a Bolus Calculator

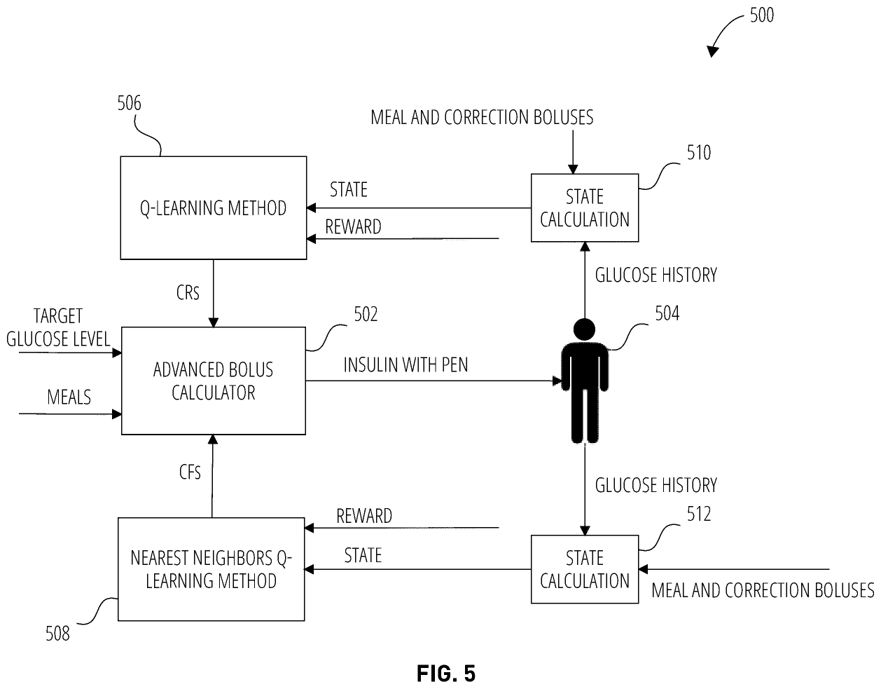

FIG. 5 is a block diagram of an architecture of a system 500 for an advanced bolus calculator 502 for PWDs (e.g., individual 504) with type 1 diabetes on MDI therapy, according to some embodiments. Methods discussed above (e.g., learning method 200, learning method 300, method 400) may (e.g., at the end of each day, without limitation) use glucose, insulin, and meal data to compute next-day's CRs and CFs (e.g., optimal CRs and CFs) utilizing Algorithms 1 and 2 or utilizing learning method 200 and learning method 300.

The system 500 includes, in addition to the advanced bolus calculator 502 and the individual 504, a Q-learning method 506, a nearest-neighbors Q-learning method 508, a state calculation 510, and a state calculation 512. By way of non-limiting example, the Q-learning method 506 may include the learning method 200 of FIG. 2. Also by way of non-limiting example, the Q-learning method 506 may include Algorithm 1, which is discussed above. By way of non-limiting example, the nearest-neighbors Q-learning method 508 may include the learning method 300 of FIG. 3. Also by way of non-limiting example, the nearest-neighbors Q-learning method 508 may include Algorithm 2, which is discussed above.

The state calculation 510 determines a state responsive to a glucose history received for the individual 504 and data corresponding to meal and correction boluses. A state may be determined responsive to one or more of glucose data, insulin data, or meal data. The state calculation 510 delivers the determined state to the Q-learning method 506. The Q-learning method 506 determines CRs responsive to the determined state from the state calculation 510 and a determined reward. The Q-learning method 506 provides the CRs to the advanced bolus calculator 502.

The state calculation 512 determines a state responsive to the glucose history received for the individual 504 and data corresponding to meal and correction boluses. The state calculation 512 delivers the determined state to the nearest-neighbors Q-learning method 508. The nearest-neighbors Q-learning method 508 determines CFs responsive to the determined state from the state calculation 512 and a determined reward. The nearest-neighbors Q-learning method 508 provides the CFs to the advanced bolus calculator 502.

Responsive to the CRs, the CFs, a target glucose level (e.g., determined by a healthcare professional, without limitation), and data corresponding to meals, the advanced bolus calculator 502 determines a bolus dose. Insulin at substantially the determined bolus dose may be delivered to the individual 504 (e.g., utilizing an injection pen, utilizing an insulin pump, without limitation).

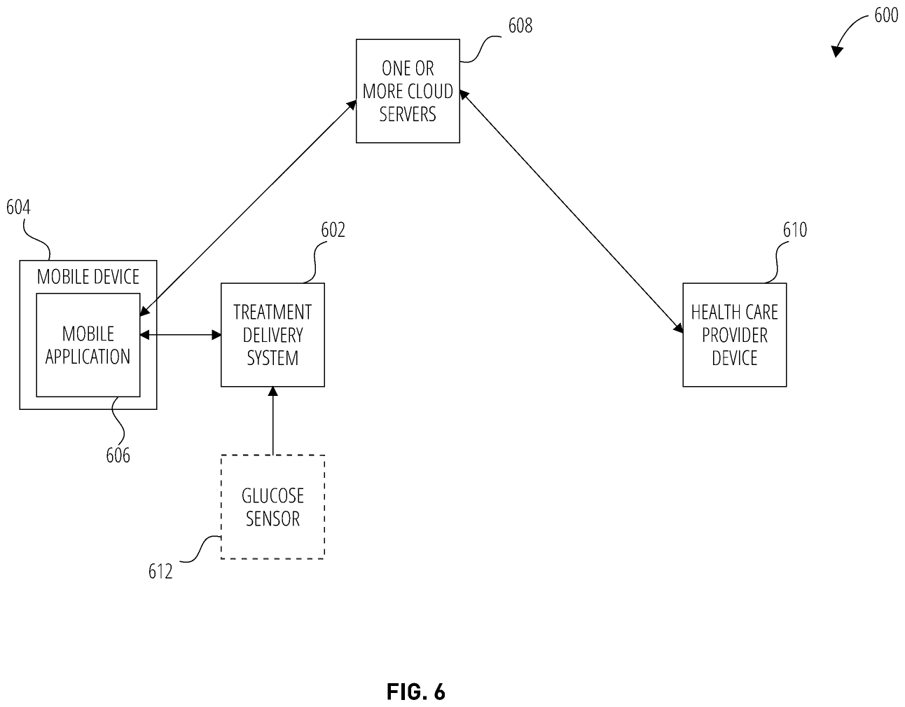

FIG. 6 is a block diagram of a system 600, according to some embodiments. The system 600 includes a treatment delivery system 602, a mobile device 604, one or more cloud servers 608, a health care provider device 610, and in some embodiments, a glucose sensor 612. The glucose sensor may include an in vivo glucose sensor having a first portion configured to be placed under the skin and a second portion configured to be arranged above the skin. The second portion may be coupled to sensor electronics, including one or more of a power source, processors, and communication circuitry, such as transceiver for communicating with another component, e.g., mobile device 604. The communication may be by a wireless communication protocol. Portions of the system 500 of FIG. 5 may be executed by one or more of the treatment delivery system 602, the mobile device 604, the one or more cloud servers 608, and the health care provider device 610.

By way of non-limiting example, the advanced bolus calculator 502 may be performed by the treatment delivery system 602. Also by way of non-limiting example, the treatment delivery system 602 may be performed by a mobile application 606 executed by the mobile device 604 or at the one or more cloud servers 608, then provided to the treatment delivery system 602 by the mobile device 604. As a specific, non-limiting example, the one or more cloud servers 608 may perform the Q-learning method 506, the nearest-neighbors Q-learning method 508, the state calculation 510, and the state calculation 512, and deliver the CRs and CFs to the treatment delivery system 602 via the mobile application 606. As another specific, non-limiting example, if the treatment delivery system 602 has sufficient processing power, the treatment delivery system 602 may perform operations corresponding to the Q-learning method 506, the nearest-neighbors Q-learning method 508, the state calculation 510, and the state calculation 512.

The treatment delivery system 602 may include one or more injection pens, an insulin pump, or other treatment delivery system. By way of non-limiting example, the treatment delivery system 602 may include injection pens including caps that include electronics to enable the treatment delivery system 602 to communicate with the glucose sensor 612 and a mobile application 606 executed by the mobile device 604. In some such examples the glucose sensor 612 may detect glucose levels in an individual (e.g., individual 504 of FIG. 5, without limitation) and provide the detected glucose levels with corresponding time stamps to the treatment delivery system 602 (e.g., via an RFID scanner of treatment delivery system 602). The treatment delivery system 602 may interact with the mobile application 606 to deliver this glucose information to the mobile device 604. Alternatively, the glucose information may be obtained by the individual via blood tests and manual entry into an interface of the treatment delivery system 602 or the mobile application 606.

In some embodiments the treatment delivery system 602 may also be configured to capture data corresponding to meals. By way of non-limiting example, the treatment delivery system 602 may include injection pens including caps that include electronics to enable the treatment delivery system 602 to detect removal of a lid over a needle of the pen to detect a use of the pen to deliver an injection prior to a meal. The electronics may detect a time of the removal of the lid, and estimate a number of calories for the meal based on the time of day (e.g., if in the morning, a breakfast-sized dose, if near noon, a lunch-sized dose, or if in the evening, a dinner-sized dose). Also by way of non-limiting example, the pen cap or the mobile application 606 may include an interface that enables the individual to enter an estimate of how many calories they are to consume in the upcoming meal. For example, options such as “small,” “medium,” and “large,” may be provided, and the user may select the one of the options, each corresponding to a number of carbohydrates. As another example, a list selectable carbohydrate numbers may be presented to the individual, and the individual may select the carbohydrate number that is estimated to best fit the upcoming meal.

In some embodiments the health care provider device 610 may enable a health care professional to enter information such as a target glucose level, which may be communicated through the medical treatment system 600 to whichever device will ultimately calculate the bolus. For example, if the treatment delivery system 602 will ultimately be used to calculate the bolus, the health care provider device 610 may be used to program the treatment delivery system 602 with the target glucose level.

### G. Baseline Learning Methods

The learning methods discussed above were compared with the run-to-run algorithm and Herero et al.'s algorithm. In the run-to-run algorithm, the CRs for each main meal m (breakfast, lunch, and dinner) are adjusted as follows:

$${CR}_{k + 1} = \{ \begin{matrix}{CR}_{k} + 0.15 {CR}_{m} {P}_{k}^{hypo} & if {P}_{k}^{hypo} > 0 \% \\ {CR}_{k} - {CR}_{m} ( 0.05 {P}_{k}^{hyper} + 0.05 \frac{{G}_{mean} - 6.4}{{G}_{T}} ) & if {P}_{k}^{hypo} = 0 \%\end{matrix} \qquad ( 25 )$$

where CRₘ is the initial CR of a meal m, Pₖ^{hypo} and Pₖ^{hyper} are the percentage of time in hypoglycemia and hyperglycemia in the period seven hours after the meal (or until next meal if it is within seven hours), and Gₘₑₐₙ is the post-prandial mean glucose level in the period seven hours (or until next meal if it is within seven hours).

In Herero et al.'s algorithm, the CRs for each meal m are adjusted as follows:

$${CR}_{k + 1} = \frac{CHO + \frac{{G}_{m} - {G}_{T}}{( \frac{108}{5.7 W} )}}{B + I O B + {B}_{add}} \qquad ( 26 )$$

where CHO is the amount of meal's carbohydrate, Gₘ is the mealtime glucose level, G_{T} is the target glucose level, W is the weight of the individual in kilogram (kg), B is the recommended insulin dose at mealtime, IOB denotes the insulin on board, B_{add} is defined as:

$${B}_{add} = \frac{{G}_{min}^{h} - {G}_{t}}{CF} \qquad ( 27 )$$

and Gₘᵢₙ^{h} is the minimum glucose level in the period between two and five hours after the meal.

For the baseline algorithms, CF was calculated utilizing the static 100-rule equation.

### H. In-Silico Environment

The in-silico environment included PWDs based on Hovorka's model of R. Hovorka et al., “Partitioning glucose distribution/transport, disposal, and endogenous production during IVGTT,” Am J Physiol Endocrinol Metab, vol. 282, no. 5, pp. e992-e1007, 2002. Parameters in the model were sampled from a log-normal distribution with their mean values obtained from Wilinska et al., “Simulation environment to evaluate closed-loop insulin delivery systems in type 1 diabetes,” J Diabetes Sci Technol, vol. 4, no. 1, pp. 132-44, 2010, and correlations from healthy individual data.

Inter-day glucose variability was added by making model parameters for each individual oscillate periodically, with random phase and frequency, and by adding daily random insulin and glucose fluxes. Variability in meal absorption, explaining fast and slow carbohydrate meals, was implemented by randomly varying the time-to-peak of the meal absorption time (as discussed in A. Haidar et al., “Stochastic Virtual Population of Subjects With Type 1 Diabetes for the Assessment of Closed-Loop Glucose Controllers,” IEEE Trans Biomed Eng, vol. 60, no. 12, pp. 3524-33, 2013). The simulation environment included noise in glucose measurements with a correlation of 80% and a coefficient of variation of 7% (see A. Facchinetti et al., “Modeling the glucose sensor error,” IEEE Trans Biomed Eng, vol. 61, no. 3, pp. 620-9, 2014).

Each in-silico individual possesses unique and optimal CRs and long-acting basal insulin dose. A clinical dataset of 81 individuals was used to validate the simulator.

The feasibility of the learning methods disclosed herein was evaluated on 100 in-silico PWDs over 8 weeks. Each in-silico individual was initialized with nonoptimal values of CRs and CF, by imposing uniform random errors by 30-70% on the optimal CRs and CFs. Each in-silico individual ate 15 g of carbohydrate whenever glucose level dropped below 4.0 mmol/L, and this was repeated every 15 min until glucose levels increase above 4.0 mmol/L.

The learning algorithm was tested in two scenarios: nominal and variance. In the nominal scenario, each in-silico individual consumed a breakfast (at 7:00 a.m.), a lunch (at 2:00 p.m.), and a dinner (at 8:00 p.m.) of 40 g, 60 g, and 80 g of carbohydrate, respectively. In the variance scenario, random carbohydrate counting errors (with zero mean and coefficient of variation of 40%) and random variations were added to consumed meal sizes and times as shown by Table I.

TABLE I
Meal plan for the variance scenario (uniform distributions)

| | Breakfast | Lunch | Dinner | Snack |
|---|---|---|---|---|
| Time | 7:00 a.m.-8:00 | 12:00 p.m.-1:00 | 6:00 p.m.-7:30 | 8:30 p.m.-9:30 |
| | a.m. | p.m. | p.m. | p.m. |
| CHO (gram) | [30, 50] | [60, 80] | [50, 70] | [10, 15] |

Daytime correction boluses were generated if (i) the glucose level was ≥10 mmol/L at least 154 (90-205) minutes after the meal (e.g., due to carbohydrate counting errors, without limitation), resembling data reported from two studies containing 49,995 days, 296,685 meals (including snacks), and 61,654 insulin correction boluses (ii) the post-snack glucose level was ≥10 mmol/L for at least 30 minutes (iii) or the glucose level was ≥10 mmol/L due to missing boluses of 2.1 (0-9) per week. Similarly, nighttime correction boluses were generated if the glucose level was ≥10 mmol/L or if the post-snack glucose level is ≥10 mmol/L for at least 30 minutes. In real time, these nighttime correction boluses might occur due to suboptimal basal dose, dawn phenomenon, daytime physical activity, nighttime snack or suboptimal dinner boluses.

### III. Results from Experimentation

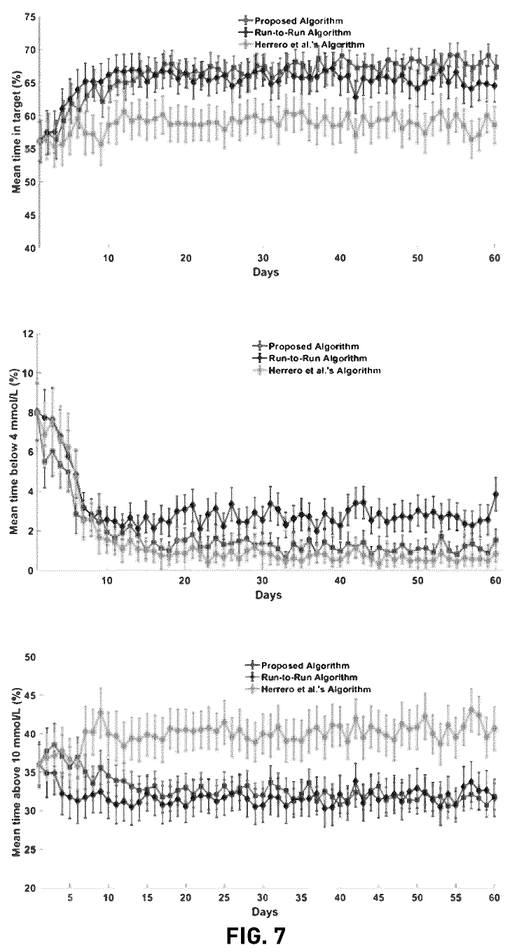

FIG. 7 illustrates a percentage time spent in target range, below 4.0 mmol/L, and above 10.0 mmol/L with the nominal scenario (mean±SD). The glycemic outcomes from the three algorithms (the disclosed algorithm, the run-to-run algorithm, and Herrero et. al's algorithm) for both the nominal and variance scenarios are reported. Before starting the sixty-day simulation, initial seven-day were simulated for calculating baseline outcomes.

The tuning parameters were selected as t₁=t₃=2.5 hours, t₂=t₂=6 hours,

$${ROC}_{T} = \frac{0.02 mmol}{L min} , and {E}_{T} = {E}_{c} = 1 \frac{mmol}{L} .$$

The intuition behind the chosen values of t_{i|i=1, 2, 3, 4} and ROC_{T} is to account for the variability due to different profiles of meal and insulin absorptions (e.g., low and high glycemic index foods). The intuition behind the chosen thresholds

$${E}_{T} = {E}_{c} = 1 \frac{mmol}{L}$$

is to make the learning algorithm's updates robust in face of disturbances such as carbohydrate counting errors, delayed insulin boluses, and metabolic variability.

Nominal scenario: FIG. 7 shows changes in glycemic outcomes for 100 in-silico individuals over sixty days with methods disclosed herein and the baseline algorithms. Comparing the last week of the sixty-day simulation to baseline, the mean time spent in the target range increased from 55% to 68% with embodiments disclosed herein compared to 64% and 58% with the run-to-run and Herrero et. al's algorithms, respectively. The mean time spent below 4 mmol/L decreased from baseline of 8% to 0.8% with embodiments disclosed herein compared to 2.8% with the run-to-run algorithm and 0.5% with Herrero et. al's algorithm. The mean time spent above 10 mmol/L decreased from baseline of 37% to 31% with embodiments disclosed herein compared to 33% with the run-to-run algorithm; the mean time spent above 10 mmol/L increased to 41% with Herrero et. al's algorithm. Table II and Table III report glycemic outcomes including overall, daytime, nighttime, post-meal, and post-correction periods.

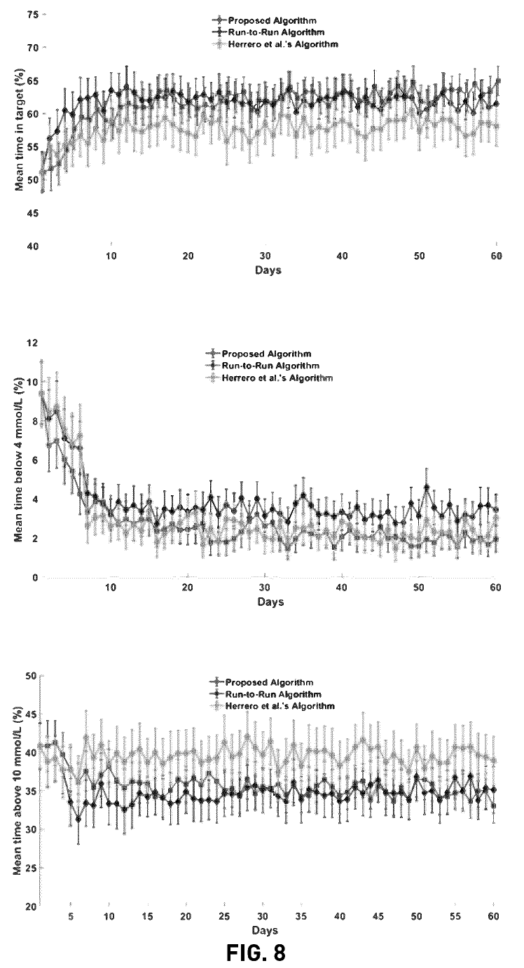

FIG. 8 illustrates a percentage time spent in target range, below 4.0 mmol/L, and above 10.0 mmol/L with the variance scenario (mean±SD). FIG. 8 shows changes in glycemic outcomes for 100 in-silico individuals over sixty days with embodiments disclosed herein and the baseline algorithms. Comparing the last week of the sixty-day simulation to baseline, the mean time spent in the target range increased from 51% to 64% with embodiments disclosed herein compared to the 61% and 58% with the run-to-run and Herrero et. al's algorithms, respectively. The mean time spent below 4 mmol/L decreased from baseline of 9% to 1.9% with embodiments disclosed herein compared to 3.4% with the run-to-run algorithm and 2.3% with Herrero et. al's algorithm. The mean time spent above 10 mmol/L decreased from baseline of 40% to 34% with embodiments disclosed herein compared to 35% with the run-to-run algorithm; the mean time spent above 10 mmol/L did not change from the baseline with Herrero et. al's algorithm. Table II and Table III report glycemic outcomes including overall, daytime, nighttime, post-meal, and post-correction periods.

TABLE II
Summary of glycemic outcomes in the nominal and variance scenarios.

| | | | | | Herrero | |
|---|---|---|---|---|---|---|
| | | | | Run-to-run | et. al's | Disclosed |
| Scenario | Period | Outcomes | Baseline | algorithm | algorithm | algorithm |
| Nominal | Overall | Time in | 55(15) | 64 (12) | 58 (14) | 68 (9) |
| | | target 4-10 | | | | |
| | | mmol/L | | | | |
| | | Time below | 8 (8) | 2.8 (4.3) | 0.5 (2.2) | 0.8 (2.8) |
| | | 4 mmol/L | | | | |
| | | Time above | 37 (14) | 33 (12) | 41 (14) | 31 (8.7) |
| | | 10 mmol/L | | | | |
| | Daytime | Time in | 60 (17.3) | 68 (10) | 62 (10.5) | 67.5 (7) |
| | (7 AM to | target 4-10 | | | | |
| | 12 AM) | mmol/L | | | | |
| | | Time below | 5.4 (6.7) | 3.5 (2.4) | 0.8 (1.4) | 1.2 (1.5) |
| | | 4 mmol/L | | | | |
| | | Time above | 34.7 (15) | 28.5 (11) | 37.2 (12) | 31.3 (9.2) |
| | | 10 mmol/L | | | | |
| | Nighttime | Time in | 44 (28.9) | 57 (15.4) | 51 (16) | 67 (10.4) |
| | (7 AM to | target 4-10 | | | | |
| | 12 AM) | mmol/L | | | | |
| | | Time below | 8.6 (13.9) | 1.4 (2.3) | 0.7 (0.9) | 1.2 (1.5) |
| | | 4 mmol/L | | | | |
| | | Time above | 47.5 (16) | 41.7 (12) | 48 (15) | 31 (8) |
| | | 10 mmol/L | | | | |
| Variance | Overall | Time in | 51 (16) | 61 (16) | 58 (15) | 64 (11) |
| | | target 4-10 | | | | |
| | | mmol/L | | | | |
| | | Time below | 9 (7.9) | 3.4 (4) | 2.3 (4.3) | 1.9 (3) |
| | | 4 mmol/L | | | | |
| | | Time above | 40 (16) | 35 (15) | 40 (16) | 34 (11) |
| | | 10 mmol/L | | | | |
| | Daytime | Time in | 53 (18) | 62 (12) | 59 (10) | 63 (8.4) |
| | (7 AM to | target 4-10 | | | | |
| | 12 AM) | mmol/L | | | | |
| | | Time below | 7.6 (8) | 4.1 (2.3) | 3 (2.6) | 2.5 (2.3) |
| | | 4 mmol/L | | | | |
| | | Time above | 39.4 (17) | 34 (14) | 38 (18) | 34 (12) |
| | | 10 mmol/L | | | | |
| | Nighttime | Time in | 46 (28) | 58 (16) | 54 (16) | 66 (10) |
| | (7 AM to | target 4-10 | | | | |
| | 12 AM) | mmol/L | | | | |
| | | Time below | 9 (14) | 1.8 (2.8) | 0.9 (1.6) | 0.9 (1.5) |
| | | 4 mmol/L | | | | |
| | | Time above | 45 (14) | 40 (14) | 45 (17) | 33 (11) |
| | | 10 mmol/L | | | | |

Results are mean (SD).

### IV. Discussion of a Bolus Calculator

PWDs live with their life-long burden of managing their glucose levels. With the rapid advances in glucose sensors technology and smart insulin pens, it has become possible to automatically adjust their therapy parameters (e.g., CRs. CFs, and basal doses) through the use of algorithms. Disclosed herein is an advanced bolus calculator for individuals on MDI therapy that adjusts their CRs and CFs by analyzing their glucose and insulin data.

The disclosed bolus calculator is designed utilizing the Q-learning approach to adjust CRs and utilizing the nearest-neighbors Q-learning approach to adjust CFs. Separate CFs for daytime and nighttime are disclosed to account for diurnal changes in insulin sensitivity. To assess the performance of the disclosed bolus calculator, an in-silico environment was used to challenge it by (i) adding random carbohydrate counting errors of zero mean and 40% coefficient of variation, and (ii) utilizing random meal sizes and random meal ingestion times. Despite this, the disclosed bolas calculator improved time in target and reduced hypoglycemia.

Compared to the Herrero et al.'s algorithm, the disclosed methods achieved a greater reduction in hypoglycemia during the daytime as well the overall 24 hour period, both in nominal and variance scenarios (Table II). Moreover, the disclosed methods achieved a greater time in target during the nighttime and 24 hour periods, in both the nominal and variance scenarios (Table II). These benefits with the disclosed methods be explained by multiple factors (i) the disclosed methods adjust daytime and nighttime CFs separately and (ii) the disclosed methods use novel state and reward functions that were designed to promote early and safe convergence.

Given the increasing incidence of diabetes and the increasing deficiency of expert endocrinologists, the responsibility to manage PWDs may be increasingly shifted to primary-care doctors. As some doctors may be naïve to the use of glucose sensor and smart insulin pens, the disclosed bolus calculator might help in making insulin dosing decisions efficiently in primary-care settings. Moreover, the disclosed algorithm may be utilized to propose more frequent dosing adjustments compared to physicians (e.g., weekly vs every three to six months).

The disclosed methods may have several limitations. First, the disclosed bolus calculator is the assumption that meal insulin bolus is calculated based on the carbohydrate content alone. Studies have shown that carbohydrate-matched high-fat meals require higher insulin doses and can lead to prolonged hyperglycemia for up to five hours after meal. Second, although the above-discussed in-silico study demonstrated improvements in glycemic outcomes, simulations have their own downsides. Simulations tend to over-estimate the benefits of interventions as they do not take into account all the perturbations and uncertainties that exist in real-world settings.

### V. Conclusion

Methods, and related systems and devices, are disclosed to automatically adjust the parameters of an insulin bolus calculator for PWDs on MDI therapy. The disclosed methods were developed utilizing the reinforcement learning with nearest-neighbors method applied to continuous glucose monitoring and insulin data. The disclosed methods were tested on 100 in-silico individuals. Simulation results support effectiveness of the methods in improving glucose control.

TABLE III
Summary of glycemic outcomes in the post-
meal and post-correction boluses periods.

| | | | | | Herrero | |
|---|---|---|---|---|---|---|
| | | | | Run-to-run | et. al's | disclosed |
| Scenario | Period | Outcomes | Baseline | algorithm | algorithm | algorithm |
| Nominal | Post-meal | Time in | 54 (18) | 62 (8) | 53 (9) | 60 (8) |
| | boluses | target 4-10 | | | | |
| | | mmol/L | | | | |
| | | Time below | 4.5 (5.2) | 2 (1.6) | 0.4 (0.8) | 0.6 (0.9) |
| | | 4 mmol/L | | | | |
| | | Time above | 40 (16) | 36 (11) | 47 (8) | 39 (9) |
| | | 10 mmol/L | | | | |
| | Post- | Time in | | | | |
| | correction | target 4-10 | 59 (15) | 62 (10) | 57 (15) | 68 (7) |
| | boluses | mmol/L | | | | |
| | | Time below | 9.9 (14.3) | 1.3 (1.7) | 0.3 (0.9) | 0.7 (1) |
| | | 4 mmol/L | | | | |
| | | Time above | 31 (20) | 37 (13) | 43 (17) | 31 (5) |
| | | 10 mmol/L | | | | |
| Variance | Post-meal | Time in | 45 (16) | 53 (10) | 50 (8) | 53 (9) |
| | boluses | target 4-10 | | | | |
| | | mmol/L | | | | |
| | | Time below | 6.9 (6.9) | 3.2 (1.8) | 2.4 (1.8) | 1 (1.2) |
| | | 4 mmol/L | | | | |
| | | Time above | 48 (15) | 46 (9) | 48 (10) | 46 (8) |
| | | 10 mmol/L | | | | |
| | Post- | Time in | 53 (15) | 60 (15) | 58 (14) | 63 (8) |
| | correction | target 4-10 | | | | |
| | boluses | mmol/L | | | | |
| | | Time below | 10 (14.4) | 2 (2.4) | 0.9 (2) | 0.8 (1.2) |
| | | 4 mmol/L | | | | |
| | | Time above | 37 (18) | 38 (17) | 41 (12) | 36 (10) |
| | | 10 mmol/L | | | | |

Results are mean (SD).

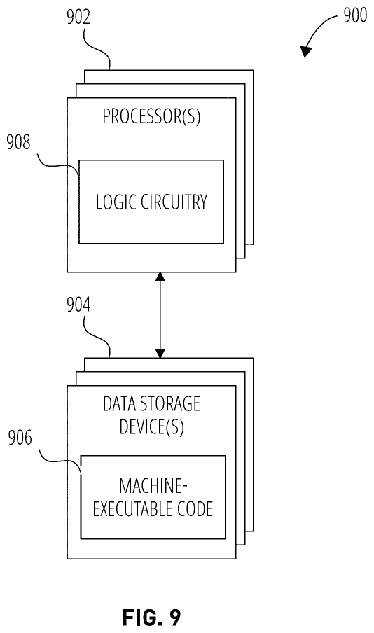

It will be appreciated by those of ordinary skill in the art that functional elements of embodiments disclosed herein (e.g., functions, operations, acts, processes, and/or methods) may be implemented in any suitable hardware, software, firmware, or combinations thereof. FIG. 9 illustrates non-limiting examples of implementations of functional elements disclosed herein. In some embodiments, some or all portions of the functional elements disclosed herein may be performed by hardware specially configured for carrying out the functional elements.

FIG. 9 is a block diagram of circuitry 900 that, in some embodiments, may be used to implement various functions, operations, acts, processes, and/or methods disclosed herein. The circuitry 900 includes one or more processors 902 (sometimes referred to herein as “processors 902”) operably coupled to one or more data storage devices (sometimes referred to herein as “storage 904”). The storage 904 includes machine-executable code 906 stored thereon and the processors 902 include logic circuitry 908. The machine-executable code 906 includes information describing functional elements that may be implemented by (e.g., performed by) the logic circuitry 908. The logic circuitry 908 is adapted to implement (e.g., perform) the functional elements described by the machine-executable code 906. The circuitry 900, when executing the functional elements described by the machine-executable code 906, should be considered as special purpose hardware configured for carrying out functional elements disclosed herein. In some embodiments the processors 902 may be configured to perform the functional elements described by the machine-executable code 906 sequentially, concurrently (e.g., on one or more different hardware platforms), or in one or more parallel process streams.

When implemented by logic circuitry 908 of the processors 902, the machine-executable code 906 is configured to adapt the processors 902 to perform operations of embodiments disclosed herein. For example, the machine-executable code 906 may be configured to adapt the processors 902 to perform at least a portion or a totality of the method 100 of FIG. 1, the learning method 200 of FIG. 2, the learning method 300 of FIG. 3, the method 400 of FIG. 4, the Q-learning method 506 of FIG. 5, the nearest-neighbors Q-learning method 508 of FIG. 5, the state calculation 510 of FIG. 5, the state calculation 512 of FIG. 5, Algorithm 1, Algorithm 2, and/or Algorithm 3. As another example, the machine-executable code 906 may be configured to adapt the processors 902 to perform at least a portion or a totality of the operations discussed for the treatment delivery system 602 of FIG. 6, the mobile application 606 of FIG. 6, the one or more cloud servers 608 of FIG. 6, and/or the health care provider device 610 of FIG. 6.

The processors 902 may include a general purpose processor, a special purpose processor, a central processing unit (CPU), a microcontroller, a programmable logic controller (PLC), a digital signal processor (DSP), an application specific integrated circuit (ASIC), a field-programmable gate array (FPGA) or other programmable logic device, discrete gate or transistor logic, discrete hardware components, other programmable device, or any combination thereof designed to perform the functions disclosed herein. A general-purpose computer including a processor is considered a special-purpose computer while the general-purpose computer is configured to execute functional elements corresponding to the machine-executable code 906 (e.g., software code, firmware code, hardware descriptions) related to embodiments of the present disclosure. It is noted that a general-purpose processor (may also be referred to herein as a host processor or simply a host) may be a microprocessor, but in the alternative, the processors 902 may include any conventional processor, controller, microcontroller, or state machine. The processors 902 may also be implemented as a combination of computing devices, such as a combination of a DSP and a microprocessor, a plurality of microprocessors, one or more microprocessors in conjunction with a DSP core, or any other such configuration.

In some embodiments the storage 904 includes volatile data storage (e.g., random-access memory (RAM)), non-volatile data storage (e.g., Flash memory, a hard disc drive, a solid state drive, erasable programmable read-only memory (EPROM), etc.). In some embodiments the processors 902 and the storage 904 may be implemented into a single device (e.g., a semiconductor device product, a system on chip (SOC), etc.). In some embodiments the processors 902 and the storage 904 may be implemented into separate devices.

In some embodiments the machine-executable code 906 may include computer-readable instructions (e.g., software code, firmware code). By way of non-limiting example, the computer-readable instructions may be stored by the storage 904, accessed directly by the processors 902, and executed by the processors 902 utilizing at least the logic circuitry 908. Also by way of non-limiting example, the computer-readable instructions may be stored on the storage 904, transferred to a memory device (not shown) for execution, and executed by the processors 902 utilizing at least the logic circuitry 908. Accordingly, in some embodiments the logic circuitry 908 includes electrically configurable logic circuitry 908.

In some embodiments the machine-executable code 906 may describe hardware (e.g., circuitry) to be implemented in the logic circuitry 908 to perform the functional elements. This hardware may be described at any of a variety of levels of abstraction, from low-level transistor layouts to high-level description languages. At a high-level of abstraction, a hardware description language (HDL) such as an IEEE Standard hardware description language (HDL) may be used. By way of non-limiting examples, VERILOG™, SYSTEMVERILOG™ or very large-scale integration (VLSI) hardware description language (VHDL™) may be used.

HDL descriptions may be converted into descriptions at any of numerous other levels of abstraction as desired. As a non-limiting example, a high-level description can be converted to a logic-level description such as a register-transfer language (RTL), a gate-level (GL) description, a layout-level description, or a mask-level description. As a non-limiting example, micro-operations to be performed by hardware logic circuits (e.g., gates, flip-flops, registers, without limitation) of the logic circuitry 908 may be described in a RTL and then converted by a synthesis tool into a GL description, and the GL description may be converted by a placement and routing tool into a layout-level description that corresponds to a physical layout of an integrated circuit of a programmable logic device, discrete gate or transistor logic, discrete hardware components, or combinations thereof. Accordingly, in some embodiments the machine-executable code 906 may include an HDL, an RTL, a GL description, a mask level description, other hardware description, or any combination thereof.

In embodiments where the machine-executable code 906 includes a hardware description (at any level of abstraction), a system (not shown, but including the storage 904) may be configured to implement the hardware description described by the machine-executable code 906. By way of non-limiting example, the processors 902 may include a programmable logic device (e.g., an FPGA or a PLC) and the logic circuitry 908 may be electrically controlled to implement circuitry corresponding to the hardware description into the logic circuitry 908. Also by way of non-limiting example, the logic circuitry 908 may include hard-wired logic manufactured by a manufacturing system (not shown, but including the storage 904) according to the hardware description of the machine-executable code 906.

Regardless of whether the machine-executable code 906 includes computer-readable instructions or a hardware description, the logic circuitry 908 is adapted to perform the functional elements described by the machine-executable code 906 when implementing the functional elements of the machine-executable code 906. It is noted that although a hardware description may not directly describe functional elements, a hardware description indirectly describes functional elements that the hardware elements described by the hardware description are capable of performing.

As used in the present disclosure, the terms “module” or “component” may refer to specific hardware implementations configured to perform the actions of the module or component and/or software objects or software routines that may be stored on and/or executed by general purpose hardware (e.g., computer-readable media, processing devices, etc.) of the computing system. In some embodiments, the different components, modules, engines, and services described in the present disclosure may be implemented as objects or processes that execute on the computing system (e.g., as separate threads). While some of the system and methods described in the present disclosure are generally described as being implemented in software (stored on and/or executed by general purpose hardware), specific hardware implementations or a combination of software and specific hardware implementations are also possible and contemplated.

As used in the present disclosure, the term “combination” with reference to a plurality of elements may include a combination of all the elements or any of various different subcombinations of some of the elements. For example, the phrase “A, B, C, D, or combinations thereof” may refer to any one of A, B, C, or D; the combination of each of A, B, C, and D; and any subcombination of A, B, C, or D such as A, B, and C; A, B, and D; A, C, and D; B, C, and D; A and B; A and C; A and D; B and C; B and D; or C and D.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

While the present disclosure has been described herein with respect to certain illustrated embodiments, those of ordinary skill in the art will recognize and appreciate that the present invention is not so limited. Rather, many additions, deletions, and modifications to the illustrated and described embodiments may be made without departing from the scope of the invention as hereinafter claimed along with their legal equivalents. In addition, features from one embodiment may be combined with features of another embodiment while still being encompassed within the scope of the invention as contemplated by the inventor.

Exemplary embodiments are set forth in the following numbered clauses:
1. A system for determining an insulin dose, the system comprising one or more computer-readable storage media having computer-readable instructions stored thereon, the computer-readable instructions configured to instruct the one or more processors to:
   determine a state based on glucose, meal and insulin information of a user;
   track variations in carbohydrate ratios (CRs) utilizing a Q-learning algorithm based on the determined state;
   track variations in correction factors (CFs) utilizing a nearest-neighbors Q-learning algorithm based on the determined state; and
   determine a dose for a meal bolus, correction bolus, or basal, responsive to the tracked CRs and the tracked CFs.
2. The system of clause 1, wherein the system is configured to instruct the one or more processors to track the variations in the CRs by initializing an action value function, choosing actions from the current state, obtaining a CR value based on the chosen action, and determining a reward for the obtained CR value.
3. The system of clause 1, wherein the computer-readable instructions are configured to instruct the one or more processors to track variations in the CRs by:
   initializing a discount factor that weights a preference of immediate over future rewards, a learning rate, and an ε-greedy policy probability;
   initializing an action-value function utilizing clinical guidelines;
   evaluating a current state; and
   repeating the following operations:
      choosing an action from the current state utilizing an ε-greedy policy;
      obtaining, from the chosen action, a CR value;
      applying the obtained CR value to observe a reward and a next state;
      updating the action-value function; and
      evaluating a next state.
4. The system of any preceding clause, wherein the computer-readable instructions are configured to instruct the one or more processors to track the variations in CFs utilizing the nearest-neighbors Q-learning algorithm by:
   initializing a discount factor that weights a preference of immediate over future rewards and an ε-greedy policy probability;
   constructing a finite set of a state space;
   initializing an action-value function utilizing clinical guidelines;
   setting, for each state-action pair, a counter value to zero, the counter value indicating a number of times the corresponding state-action pair has been visited;
   evaluating a current state; and
   repeating the following operations:
      choosing an action from the current state utilizing an ε-greedy policy;
      obtaining a CF value from the chosen action;
      applying the obtained CF value to observe a reward and a next state;
      determining, for each state nearest to the current state, a joint nearest-neighbors Q-value operator and incrementing the corresponding counter value by one; and
      determining, for each state nearest to the current state, an updated action-value function and a next learning rate.
5. The system of any preceding clause, wherein the computer-readable instructions are configured to estimate a basal dose.
6. The system of clause 5, wherein the computer-readable instructions are configured to estimate the basal dose by:
   initializing a discount factor that weights a preference of immediate over future rewards, a learning rate, and an ε-greedy policy probability;
   initializing an action-value function utilizing clinical guidelines;
   evaluating a current state; and
   repeating the following operations:
      choosing an action from the current state utilizing an ε-greedy policy;
      obtaining, from the chosen action, a basal dose value;
      applying the obtained basal dose value to observe a reward and a next state;
      updating the action-value function; and
      evaluating a next state.
7. The system of any preceding clause, wherein the computer-readable instructions are configured to:
   track variations in basal or bolus dose utilizing a Q-learning algorithm.
8. The system of any preceding clause, wherein one or more of the Q-learning algorithm or the nearest neighbors Q-learning algorithm comprises state and reward functions.
9. The system of clause 8, wherein the state and reward functions are at least partially based on representations of one or more of: correction bolus effect before and after meal, postprandial glucose error determined within a specified postprandial time window, or rate of glucose level change determined within the specified postprandial time window.
10. The system of clause 9, wherein the specified postprandial time window is an optimal postprandial time window to measure glucose concentration, wherein the optimal postprandial time window is optionally predetermined.
11. A method of determining an insulin dose for a meal bolus, the method comprising:
   determining a state based on glucose, meal and insulin information of a user;
   tracking variations in carbohydrate ratios (CRs) utilizing a Q-learning algorithm based on the determined state;
   tracking variations in correction factors (CFs) utilizing a nearest-neighbors Q-learning algorithm based on the determined state; and
   determining a dose for a meal bolus responsive to the tracked CRs and the tracked CFs.
12. The method of claim 11, wherein tracking the variations in the CRs comprises: initializing an action value function, choosing actions from the current state, obtaining a CR value based on the chosen action, and determining a reward for the obtained CR value.
13. The method of clause 11, wherein tracking the variations in the CRs comprises:
   initializing a discount factor that weights a preference of immediate over future rewards, a learning rate, and an ε-greedy policy probability;
   initializing an action-value function utilizing clinical guidelines;
   evaluating a current state; and
   repeating the following operations:
      choosing an action from the current state utilizing an ε-greedy policy;
      obtaining, from the chosen action, a CR value;
      applying the obtained CR value to observe a reward and a next state;
      updating the action-value function; and
      evaluating a next state.
14. The method of any of claims 11 to 13, wherein tracking the variations in the CFs comprises:
   initializing a discount factor that weights a preference of immediate over future rewards and an ε-greedy policy probability;
   constructing a finite set of a state space;
   initializing an action-value function utilizing clinical guidelines;
   setting, for each state-action pair, a counter value to zero, the counter value indicating a number of times the corresponding state-action pair has been visited;
   evaluating a current state; and
   repeating the following operations:
      choosing an action from the current state utilizing an ε-greedy policy;
      obtaining a CF value from the chosen action;
      applying the obtained CF value to observe a reward and a next state;
      determining, for each state nearest to the current state, a joint nearest-neighbors Q-value operator and incrementing the corresponding counter value by one; and
      determining, for each state nearest to the current state, an updated action-value function and a next learning rate.
15. The method of any of clauses 10 to 14, further comprising estimating a basal dose or bolus dose.
16. The method of clause 15, wherein estimating the basal dose or bolus dose comprises:
   initializing a discount factor that weights a preference of immediate over future rewards, a learning rate, and an ε-greedy policy probability;
   initializing an action-value function utilizing clinical guidelines;
   evaluating a current state; and
   repeating the following operations:
      choosing an action from the current state utilizing an ε-greedy policy;
      obtaining, from the chosen action, a basal dose value;
      applying the obtained basal dose value to observe a reward and a next state;
      updating the action-value function; and
   evaluating a next state.
17. The method of any of clauses 10 to 16, comprising:
   tracking variations in basal or bolus dose utilizing a Q-learning algorithm.
   18. The method of any of clauses 10 to 17, wherein one or more of the Q-learning algorithm or the nearest neighbors Q-learning algorithm comprises state and reward functions.
   19. The method of clause 18, wherein the state and reward functions are at least partially based on representations of one or more of: correction bolus effect before and after meal, postprandial glucose error determined within a specified postprandial time window, or rate of glucose level change determined within the specified postprandial time window.
20. The method of clause 19, wherein the specified postprandial time window is an optimal postprandial time window to measure glucose concentration, wherein the optimal postprandial time window is optionally predetermined.

What is claimed is:

1. A system for determining an insulin dose, the system comprising one or more processors and one or more computer-readable storage media having computer-readable instructions stored thereon, the compute-readable instructions configured to instruct the one or more processors to:
determine a state based on glucose, meal and insulin information of a user;
track variations in carbohydrate ratios (CRs) utilizing a Q-learning algorithm based on the determined state;
track variations in correction factors (CFs) utilizing a nearest-neighbors Q-learning algorithm based on the determined state; and
determine a dose for a meal bolus, correction bolus, or basal, responsive to the tracked variations in CRs and the tracked variations in CFs.

2. The system of claim 1, wherein the system is configured to instruct the one or more processors to track the variations in the CRs by initializing an action value function, choosing actions from the current state, obtaining a CR value based on the chosen action, and determining a reward for the obtained CR value.

3. The system of claim 1, wherein the system is configured to instruct the one or more processors to track the variations in the CRs by:
initializing a discount factor that weights a preference of immediate over future rewards, a learning rate, and an ε-greedy policy probability;
initializing an action-value function utilizing clinical guidelines;
evaluating a current state; and
repeating the following operations:
choosing an action from the current state utilizing an ε-greedy policy;
obtaining, from the chosen action, a CR value;
applying the obtained CR value to observe a reward and a next state;
updating the action-value function; and
evaluating a next state.

4. The system of claim 1, wherein the computer-readable instructions are configured to instruct the one or more processors to track the variations in the CFs utilizing the nearest-neighbors Q-learning algorithm by:
initializing a discount factor that weights a preference of immediate over future rewards and an ε-greedy policy probability;
constructing a finite set of a state space;
initializing an action-value function utilizing clinical guidelines;
setting, for each state-action pair, a counter value to zero, the counter value indicating a number of times the corresponding state-action pair has been visited;
evaluating a current state; and
repeating the following operations:
choosing an action from the current state utilizing an ε-greedy policy;
obtaining a CF value from the chosen action;
applying the obtained CF value to observe a reward and a next state;
determining, for each state nearest to the current state, a joint nearest-neighbors Q-value operator and incrementing the corresponding counter value by one; and
determining, for each state nearest to the current state, an updated action-value function and a next learning rate.

5. The system of claim 1, wherein the computer-readable instructions are configured to estimate a basal dose.

6. The system of claim 5, wherein the computer-readable instructions are configured to estimate the basal dose by:
initializing a discount factor that weights a preference of immediate over future rewards, a learning rate, and an ε-greedy policy probability;
initializing an action-value function utilizing clinical guidelines;
evaluating a current state; and
repeating the following operations:
choosing an action from the current state utilizing an ε-greedy policy;
obtaining, from the chosen action, a basal dose value;
applying the obtained basal dose value to observe a reward and a next state;
updating the action-value function; and
evaluating a next state.

7. The system of claim 1, wherein the computer-readable instructions are configured to:
track variations in basal or bolus dose utilizing a Q-learning algorithm.

8. The system of claim 1, wherein one or more of the Q-learning algorithm or the nearest neighbors Q-learning algorithm comprises state and reward functions.

9. The system of claim 8, wherein the state and reward functions are at least partially based on representations of one or more of: correction bolus effect before and after meal, postprandial glucose error determined within a specified postprandial time window, or rate of glucose level change determined within the specified postprandial time window.

10. The system of claim 9, wherein the specified postprandial time window is an optimal postprandial time window to measure glucose concentration, wherein the optimal postprandial time window is predetermined.

11. A method of determining an insulin dose for a meal bolus, the method comprising:
determining a state based on glucose, meal and insulin information of a user;
tracking variations in carbohydrate ratios (CRs) utilizing a Q-learning algorithm based on the determined state;
tracking variations in correction factors (CFs) utilizing a nearest-neighbors Q-learning algorithm based on the determined state; and
determining a dose for a meal bolus responsive to the tracked variations in CRs and the tracked variations in CFs.

12. The method of claim 11, wherein tracking the variations in the CRs comprises: initializing an action value function, choosing actions from the current state, obtaining a CR value based on the chosen action, and determining a reward for the obtained CR value.

13. The method of claim 11, wherein tracking the variations in the CRs comprises:
initializing a discount factor that weights a preference of immediate over future rewards, a learning rate, and an ε-greedy policy probability;
initializing an action-value function utilizing clinical guidelines;
evaluating a current state; and
repeating the following operations:
choosing an action from the current state utilizing an ε-greedy policy;
obtaining, from the chosen action, a CR value;
applying the obtained CR value to observe a reward and a next state;
updating the action-value function; and
evaluating a next state.

14. The method of claim 11, wherein tracking the variations in the CFs comprises:
initializing a discount factor that weights a preference of immediate over future rewards and an ε-greedy policy probability;
constructing a finite set of a state space;
initializing an action-value function utilizing clinical guidelines;
setting, for each state-action pair, a counter value to zero, the counter value indicating a number of times the corresponding state-action pair has been visited;
evaluating a current state; and
repeating the following operations:
choosing an action from the current state utilizing an ε-greedy policy;
obtaining a CF value from the chosen action;
applying the obtained CF value to observe a reward and a next state;
determining, for each state nearest to the current state, a joint nearest-neighbors Q-value operator and incrementing the corresponding counter value by one; and
determining, for each state nearest to the current state, an updated action-value function and a next learning rate.

15. The method of claim 11, further comprising estimating a basal dose or bolus dose.

16. The method of claim 15, wherein estimating the basal dose or bolus dose comprises:
initializing a discount factor that weights a preference of immediate over future rewards, a learning rate, and an ε-greedy policy probability;
initializing an action-value function utilizing clinical guidelines;
evaluating a current state; and
repeating the following operations:
choosing an action from the current state utilizing an ε-greedy policy;
obtaining, from the chosen action, a basal dose value;
applying the obtained basal dose value to observe a reward and a next state;
updating the action-value function; and
evaluating a next state.

17. The method of claim 11, comprising: tracking variations in basal or bolus dose utilizing a Q-learning algorithm.

18. The method of claim 11, wherein one or more of the Q-learning algorithm or the nearest neighbors Q-learning algorithm comprises state and reward functions.

19. The method of claim 18, wherein the state and reward functions are at least partially based on representations of one or more of: correction bolus effect before and after meal, postprandial glucose error determined within a specified postprandial time window, or rate of glucose level change determined within the specified postprandial time window.

20. The method of claim 19, wherein the specified postprandial time window is an optimal postprandial time window to measure glucose concentration, wherein the optimal postprandial time window is predetermined.
